SEQUENCE LISTING (55 sequences)

SEQ ID NO: 1 - protein, 18 aa, synthetic construct
GRKKRRQRRRGGEDKRKT

SEQ ID NO: 2 - protein, 20 aa, synthetic construct
GRKKRRQRRRGGKEDKRKTE

SEQ ID NO: 3 - protein, 22 aa, synthetic construct
GRKKRRQRRRGGKGEDKRKTGE

SEQ ID NO: 4 - protein, 24 aa, synthetic construct
GRKKRRQRRRGGKGGEDKRKTGGE

SEQ ID NO: 5 - protein, 18 aa, synthetic construct
GRKKRRQRRRGGEDKRKT

SEQ ID NO: 6 - protein, 18 aa, synthetic construct
GRKKRRQRRRGGEDKRKT

SEQ ID NO: 7 - protein, 18 aa, synthetic construct
GRKKRRQRRRGGEDKRKT

SEQ ID NO: 8 - protein, 18 aa, synthetic construct
GRKKRRQRRRGGEDKRKT

SEQ ID NO: 9 - protein, 18 aa, synthetic construct
GRKKRRQRRRGGEDKRKT

SEQ ID NO: 10 - protein, 24 aa, synthetic construct
GRKKRRQRRRGGKRGEDKRKTGRE

SEQ ID NO: 11 - protein, 24 aa, synthetic construct
GRKKRRQRRRGGKEGEDKRKTGEE

SEQ ID NO: 12 - protein, 20 aa, synthetic construct
GRKKRRQRRRGGKEDKRKTE

SEQ ID NO: 13 - protein, 22 aa, synthetic construct
GRKKRRQRRRGGKGEDKRKTGE

SEQ ID NO: 14 - protein, 24 aa, synthetic construct
GRKKRRQRRRGGKGGEDKRKTGGE

SEQ ID NO: 15 - protein, 20 aa, synthetic construct
GRKKRRQRRRGGKEDKRKTE

SEQ ID NO: 16 - protein, 20 aa, synthetic construct
GRKKRRQRRRGGKEDKRKTE

SEQ ID NO: 17 - protein, 16 aa, synthetic construct
GRKKRRQRRREDKRKT

SEQ ID NO: 18 - protein, 16 aa, synthetic construct
GRKKRRQRRREDKRKT

SEQ ID NO: 19 - protein, 19 aa, synthetic construct
GRKKRRQRRRGGEDKRKTC

SEQ ID NO: 20 - protein, 22 aa, synthetic construct
GRKKRRQRRRGGCGEDKRKTGC

SEQ ID NO: 21 - protein, 20 aa, synthetic construct
GRKKRRQRRRGGCEDKRKTC

SEQ ID NO: 22 - protein, 22 aa, synthetic construct
GRKKRRQRRRGGCGEDKRKTGC

SEQ ID NO: 23 - protein, 18 aa, synthetic construct
GRKKRRQRRRGGADKRKT

SEQ ID NO: 24 - protein, 18 aa, synthetic construct
GRKKRRQRRRGGEAKRKT

SEQ ID NO: 25 - protein, 18 aa, synthetic construct
GRKKRRQRRRGGEDARKT

SEQ ID NO: 26 - protein, 18 aa, synthetic construct
GRKKRRQRRRGGEDKAKT

SEQ ID NO: 27 - protein, 18 aa, synthetic construct
GRKKRRQRRRGGEDKRAT

SEQ ID NO: 28 - protein, 18 aa, synthetic construct
GRKKRRQRRRGGEDKRKA

SEQ ID NO: 29 - protein, 18 aa, synthetic construct
GRKKRRQRRRGGEDKRKT

SEQ ID NO: 30 - protein, 21 aa, synthetic construct
GRKKRRQRRRGGGEDKRKTGR

SEQ ID NO: 31 - protein, 16 aa, synthetic construct
GRKKRRQRRREDKRKT

SEQ ID NO: 32 - protein, 24 aa, synthetic construct
GRKKRRQRRRGGKRGEDKRKTGRE

SEQ ID NO: 33 - protein, 24 aa, synthetic construct
GRKKRRQRRRGGKEGEDKRKTGEE

SEQ ID NO: 34 - protein, 22 aa, synthetic construct
GRKKRRQRRRGGKREDKRKTRE

SEQ ID NO: 35 - protein, 22 aa, synthetic construct
GRKKRRQRRRGGKREDKRKTRE

SEQ ID NO: 36 - protein, 24 aa, synthetic construct
GRKKRRQRRRGGKKGEDKRKTGKE

SEQ ID NO: 37 - protein, 24 aa, synthetic construct
GRKKRRQRRRGGKDGEDKRKTGDE

SEQ ID NO: 38 - protein, 24 aa, synthetic construct
GRKKRRQRRRGGKKGEDKRKTGKE

SEQ ID NO: 39 - protein, 24 aa, synthetic construct
GRKKRRQRRRGGKDGEDKRKTGDE

SEQ ID NO: 40 - protein, 21 aa, synthetic construct
GRKKRRQRRRGGEDHARGRKT

SEQ ID NO: 41 - protein, 18 aa, synthetic construct
GRKKRRQRRRGGEDKRKT

SEQ ID NO: 42 - protein, 20 aa, synthetic construct
GRKKRRQRRRGGEDHKRHKT

SEQ ID NO: 43 - protein, 20 aa, synthetic construct
GRKKRRQRRRGGKEDKRKTE

SEQ ID NO: 44 - protein, 24 aa, synthetic construct
GRKKRRQRRRGGKGGEDKRKTGGE

SEQ ID NO: 45 - protein, 20 aa, synthetic construct
GRKKRRQRRRGGKEDKRKTE

SEQ ID NO: 46 - protein, 20 aa, synthetic construct
GRKKRRQRRRGGKEDKRKTE

SEQ ID NO: 47 - protein, 24 aa, synthetic construct
GRKKRRQRRRGGKGGEDKRKTGGE

SEQ ID NO: 48 - protein, 24 aa, synthetic construct
GRKKRRQRRRGGKRGEDKRKTGRE

SEQ ID NO: 49 - protein, 26 aa, synthetic construct
GRKKRRQRRRGGKRKGEDKRKTGGDE

SEQ ID NO: 50 - protein, 26 aa, synthetic construct
GRKKRRQRRRGGKRKGEDKRKTGGRE

SEQ ID NO: 51 - protein, 26 aa, synthetic construct
GRKKRRQRRRGGKRKTEDKRKTEDGE

SEQ ID NO: 52 - protein, 19 aa, synthetic construct
GRKKRRQRRRGGCEDKRTC

SEQ ID NO: 53 - protein, 19 aa, synthetic construct
GRKKRRQRRRGGCEDKRTC

SEQ ID NO: 54 - protein, 25 aa, synthetic construct
GRKKRRQRRRGGCKRGEDKRTGREC

SEQ ID NO: 55 - protein, 22 aa, synthetic construct
GRKKRRQRRRGGCKEDKRKTEC

(19) United States
(12) Patent Application Publication
(10) Pub. No.: US 2026/0061027 A1
(43) Pub. Date: Mar. 5, 2026

(54) NASAL DELIVERY OF APIGENIN AND CYCLIC PEPTIDE INHIBITORS OF MITOCHONDRIAL FISSION

(71) Applicant: NeuroTher, LLC, Boston, MA (US)

(72) Inventors: Bhanu Pratap Jena, Bloomfield, MI (US); Won Jin Cho, Newton, MA (US); Xin Qi, Cleveland, OH (US); Guillermo G. Marmol, Boston, MA (US)

(21) Appl. No.: 18/941,875

(22) Filed: Nov. 8, 2024

Related U.S. Application Data
(63) Continuation-in-part of application No. 18/911,604, filed on Oct. 10, 2024.
(60) Provisional application No. 63/689,104, filed on Aug. 30, 2024.

(51) Int. Cl. A61K 38/17; A61K 9/00; A61K 9/107; A61K 31/352

(52) U.S. Cl. CPC A61K 38/17; A61K 9/0043; A61K 9/1075; A61K 31/352

(57) ABSTRACT

Disclosed are compositions and/or methods of use of the compositions for patients with neuronal diseases such as AD, Parkinson's, Huntington's, multiple sclerosis, and ALS. In certain embodiments flavonoids alone, or in a pharmaceutical preparation, are administered through the nasal olfactory route. In certain embodiments the flavonoid is apigenin and the neural disease is Alzheimer's. Targeted dosages may affect mitochondrial function in brain neural cells thus ameliorating a neural disease. In some embodiments a porosome complex is administered for reconstitution into a neural cell. In certain embodiments, one or more macrocyclic or linear peptides are administered.

### CROSS REFERENCE TO RELATED APPLICATIONS

This application claims the benefit of U.S. Prov. Pat. App. No. 63/689,104 filed on 30 Aug. 2024, the entirety of which is hereby incorporated by reference. This application is a further continuation-in-part of U.S. patent application Ser. No. 18/911,604 filed on 10 Oct. 2024, the entirety of which is hereby incorporated by reference.

### SUBMISSION OF SEQUENCE LISTING

The instant application contains an electronically submitted Sequence Listing formatted as a WIPO ST.26 standard.xml file. Said ST.26 file, created on 8 Nov. 2024 is named VRN0010USPPCTP and is approximately 49,298 bytes in size and is incorporated by reference herein. The content of the file is the computer readable form (CRF) of fifty-five (55) sequences as summarized below in Table 1.

### BACKGROUND

### 1. Field of the Discovery

Embodiments of the disclosure presented herein relate generally to the field of neural diseases and their treatments. Embodiments disclosed herein relate to the administration of flavonoid compounds through the nasal olfactory route for the purposes of treating a neural disease. In some embodiments the neural disease is Alzheimer's Disease (“AD”), and the flavonoid compound is apigenin. Further embodiments additionally administer a cyclic or macrocyclic peptide. Additional embodiments incorporate porosome reconstitution.

### 2. Background Information

Alzheimer's disease is a progressive neurodegenerative disorder characterized by cognitive decline and memory loss, largely driven by factors such as amyloid-beta accumulation, tau pathology, neuroinflammation, and oxidative stress. AD is the most common cause of dementia and a leading cause of morbidity and mortality in the aging population. An estimated 6.9 million Americans aged 65 and older are living with AD in 2024, a number that is projected to increase to 13.8 million by 2060. While treatments are available to mitigate AD symptoms, there is no cure currently available. AD inevitably progresses and patients die within an average of 4 to 8 years after AD diagnosis. Therefore, there is an urgent need for novel therapeutic strategies.

There is growing evidence that AD is a consequence of metabolic disorder resulting in defects in neurotransmitter release that prevent neurotransmission, leading to a loss of neuronal function and cell death in various regions of the brain.

Mitochondria are the power houses of cells. Studies report that mitochondrial fission and fusion dynamics is critical in the maintenance of mitochondrial morphology and function. Mitochondrial fission-fusion dynamics is impaired in many aging and neurodegenerative diseases such as Alzheimer's, Parkinson's, Huntington's, multiple sclerosis, and amyotrophic lateral sclerosis (ALS). Excessive mitochondrial fission, is known to result in mitochondrial dysfunction, leading to cell damage and apoptosis. Thus, there is a need to develop treatments that can target mitochondrial dysfunction.

### SUMMARY

Disclosed are compositions and/or methods of use of the compositions for patients with neuronal diseases such as AD, Parkinson's, Huntington's, multiple sclerosis, and ALS. In certain embodiments flavonoids alone, or in a pharmaceutical preparation, are administered through the nasal olfactory route. In certain embodiments the flavonoid is apigenin and the neural disease is Alzheimer's. Targeted dosages may affect mitochondrial function in brain neural cells thus ameliorating a neural disease. In some embodiments a porosome complex is administered for reconstitution into a neural cell. In certain embodiments, one or more macrocyclic or linear peptides are administered.

Certain embodiments of the disclosure comprise a a pharmaceutically acceptable preparation of a flavonoid and at least one macrocyclic peptide associated with mitochondrial fission. In certain embodiments, the composition is a nanoemulsion for nasal administration. In still other embodiments, the at least one peptide encodes a Drp1 adaptor protein or part thereof. In still other embodiments, the macrocyclic peptide is based on DA1. In still other embodiments, the macrocyclic peptide is DA44 or DA45. In still other embodiments the composition may further comprise one or more additional linear or cyclic peptides. In certain embodiments the one or more macrocyclic peptides are DA44 and DA45. In certain compositions, the flavonoid is apigenin.

Certain embodiments take the form of compositions. Such compositions may comprise apigenin and at least one DA1 based macrocyclic peptide. In certain embodiments the at least one DA1 based macrocyclic peptide is DA44 or DA45. In further embodiments the composition further comprises at least one additional pharmaceutical excipient. In still other embodiments the composition is prepared in the form of a nanoemulsion. In still other embodiments, the composition is in the form of a suspended mist, powder, or solution.

Additional embodiments of the disclosure pertain to a method comprising administering to a subject a composition in accord with any of the embodiments disclosed herein.

The preceding general areas of utility are given by way of example only and are not intended to be limiting on the scope of the present disclosure and appended claims. Additional objects and advantages associated with the compositions, methods, and processes of the present disclosure will be appreciated by one of ordinary skill in the art in light of the instant claims, description, and examples. For example, the various aspects and embodiments of the disclosure may be utilized in numerous combinations, all of which are expressly contemplated by the present description. These additional advantages objects and embodiments are expressly included within the scope of the present disclosure. The publications and other materials used herein to illuminate the background of the disclosure, and in particular cases, to provide additional details respecting the practice, are incorporated by reference.

### BRIEF DESCRIPTION OF THE DRAWINGS

The accompanying drawings, which are incorporated into and form a part of the specification, illustrate several embodiments of the present disclosure; and, together with the description, serve to explain the principles of the disclosure. The drawings are only for the purpose of illustrating an embodiment of the invention and are not to be construed as limiting the disclosure. Further objects, features and advantages of the disclosure will become apparent from the following detailed description taken in conjunction with the accompanying figures showing illustrative embodiments of the invention, in which:

FIG. 1 presents a screening of DA1 cyclic analogs. FIG. 1A illustrates the screening breakdown of the fifty-four (54) Cyclic DA1 analogs designed and synthesized. Screening assays include: ATAD3A oligomerization assay (primary assay), mitochondrial oxidative stress assay and cell viability assay (secondary assay). The positive hits (similar or superior to DA1) are shown in the Venn diagram. FIG. 1B illustrates that among the positive 21 hits from primary and secondary screens, 11 cyclic analogs (shown by red bar) significantly reduced ATAD3A-Drp1 interaction under stress condition.

FIG. 2 presents results from in vitro mouse plasma stability measurements. FIG. 2A shows results from cyclic DA1 peptide. FIG. 2B shows results from cyclic DA44 peptide. FIG. 2C shows results from cyclic DA45 peptide.

FIG. 3 illustrates the effects of cyclic DA1 analogs in AD neuronal culture. FIG. 3A illustrates mouse hippocampal HT-22 cells were treated with DA1, DA44, DA45 or control peptide TAT (1 μM each), followed by exposure to toxic Aβ (5 μM) for 8 hrs. Mitochondrial ROS was measured by staining cells with mitoSOX fluorescence probe. The intensity of mitoROS per cell was quantitated. At least 300 cells per group was counted. FIG. 3B illustrate mouse hippocampal HT-22 cells were treated with DA1, DA44, or DA45 at the indicated doses, followed by exposure to toxic Aβ (5 μM) for 16 hrs. Cell viability was assessed using the MTT assay. Data represent the mean±SE from three independent experiments.

FIG. 4 Illustrates that the combinatorial therapy of porosome reconstitution, the DA1 peptide, and the metabolic corrector, restores cell viability and reduces oxidative stress in mitochondria to normal levels in AD neurons. (A) Schematic illustration of a synapse at the nerve ending in healthy neurons, demonstrating normal mitochondrial function, energy (ATP) generation, and porosome-mediated neurosecretion. (B) Schematic illustration of a synapse at the nerve ending in AD neurons, demonstrating abnormal mitochondrial function resulting in mitochondrial fission, loss in ATP generation, and altered porosome-mediated neurosecretion. All of the defects in AD are depicted as overcome using porosome reconstitution and/or the DA1 peptide inhibitors of mitochondrial fission therapy. (C) Cell viability is significantly enhanced in Alzheimer's neurons (Ab+TAT) by porosome reconstitution either in the presence of linear or the macrocyclic DA1 peptide WX45. HT-22 cells were pretreated with porosome (175 ng per well in a 96-well plate) overnight, followed by treatment with oligomeric Aβ₁₋₄₂ peptides (5 μM) together with DA1 (linear peptide) or WX45 (DA1 cyclic peptide) (1 μM) for 40 h. Note the cell viability in Alzheimer's neurons following porosome reconstitution, demonstrate recovery to levels in healthy neurons in the presence of the circular peptide WX45. Cell viability was measured by MTT assay after 16 h of serum starvation. Data represent the mean±SEM from at least six independent experiments. Statistical significance was determined by one-way ANOVA with Tukey's post hoc test. **p<0.01; ***p<0.001. ****p<0.0001. (D) Porosome reconstitution significantly reduces mitochondrial superoxidase activity (mitoSOX) in Alzheimer's neurons (Ab+TAT) in presence of either the linear DA1 or circular DA1 (WX45) peptide. HT-22 cells were pretreated with porosome (175 ng/well in a 96-well plate) overnight. Cells were then pre-incubated with DA1 or WX45 DA1 cyclic peptide (1 μM) for 1 hour, followed by treatment with oligomeric Aβ₁₋₄₂ peptides (10 μM) for 12 hours. Mitochondrial superoxide production was assessed 1 hour after the second peptide treatment using MitoSOX staining. MitoSOX activity in Alzheimer's neurons following porosome reconstitution demonstrate recovery to much reduced levels than found in healthy neurons in the presence of the circular peptide WX45. Data are presented as mean±SEM from at least six independent experiments. Statistical significance was determined by one-way ANOVA with Tukey's post hoc test. ***p<0.001. ****p<0.0001.

FIG. 5 Illustrates that the combination therapy of the small flavonoid molecule Apigenin and the mitochondrial fission inhibiting linear DA1 peptide, results in increased cell viability and reduced mitochondrial oxidative stress to normal levels in AD neurons. (A Left) Schematic illustration of a synapse at the nerve ending in Alzheimer's neurons, demonstrating abnormal mitochondrial function, energy (ATP) generation, and porosome-mediated neurosecretion. (A Right) Schematic illustration of a synapse at the nerve ending in AD neurons following a combination of peptide and Apigenin therapy, demonstrating normalization of mitochondrial function and corrected ATP generation and neurosecretion. All of the defects in AD are depicted as overcome using Apigenin and the DA1 peptide inhibitor of mitochondrial fission. (B) Viability of mouse hippocampal HT-22 neuronal cells exposed to 5 μM toxic oligomeric Aβ₁₋₄₂ peptides mimicking Alzheimer's (Ab+TAT), is significantly enhanced when treated with 1 μM DA1 linear peptide and 5 μM or 10 μM Apigenin. Cells were treated with DA1 1 μM+Abeta 5 μM+Apigenin (5 μM or 10 μM). After 24 h, cells were washed using serum-free media, and again treated with peptides 1 μM+Abeta 5 μM+Apigenin (5 μM or 10 μM) in serum-free medium. Cell viability was measured by MTT assay at 40 h (16 h serum starved). Data represent the mean±SEM from at least six independent experiments. Statistical significance was determined by one-way ANOVA with Tukey's post hoc test. *p<0.05; **p<0.01. (C) Mitochondrial superoxidase activity (mitoSOX) in Alzheimer's neurons (Ab+TAT) is significantly reduced in presence of the linear DA1 peptide. However, not much change is observed in the presence of either 5 μM or 10 μM of Apigenin. Mouse hippocampal HT-22 neuronal cells exposed to DA1 1 μM+Apigenin (5 μM or 10 μM) for 1 h and 10 μM Abeta (oligomeric Aβ₁₋₄₂ peptides) for 12 h. Cells were again treated with DA1 1 μM+Apigenin (5 μM or 10 μM) again 1 h before staining for mitoSOX (measure mitochondrial ROS). Data represent the mean±SEM from at least six independent experiments. Statistical significance was determined by one-way ANOVA with Tukey's post hoc test. *p<0.05; **p<0.01.

FIG. 6 presents an immunoblot and immunocytochemistry demonstration of the depletion of porosome proteins, especially the t-SNARE proteins SNAP-25 and Syntaxin-1a, which are restored following peptide and porosome-reconstitution therapy. (FIG. 6A) Note that in Alzheimer's neurons (WT-APP and Swe-APP), there is undetectable levels of SNAP-25 both in total homogenates and in the immunoisolated porosome complex, immunoisolated using the t-SNARE SNAP-25 or Syntaxin-1a antibody. FIG. 6B (subfigures a-f) presents the immunocytochemistry demonstrating depletion of porosome proteins SNAP-25, Syntaxin-1A, and Na⁺/K⁺ Transporting ATPase alpha 3 (AT1A3) in Alzheimer's neurons WT-APP (Abcta), which is fully restored to normal levels following porosome-reconstitution therapy, and further enhanced in the combined exposure to the porosome and DA1 peptide. No significant increase in SNAP-25, Syntaxin-1A, or AT1A3 immunoreactivity is observed following exposure of Apigenin to the Alzheimer's neurons (+Abeta).

FIG. 7 illustrates a Combination therapy of neuronal porosome or the small flavonoid molecule Apigenin, in combination with the mitochondrial fission inhibiting DA1 peptide for AD therapy. Note that since in >90% of Alzheimer's patients olfactory dysfunction precedes cognitive decline, and since the olfactory bulb is connected via nerves to brain regions involved in learning, memory and emotion, suggests that a combinatorial DA1 peptide therapy either with Apigenin or porosome (a 15 nm biologic), delivered via the nasal route to reach the olfactory bulb, would be most effective in AD therapy, especially at the early stages of the disease

### DETAILED DESCRIPTION

Disclosed are compositions and/or methods of use of the compositions for patients with neuronal diseases such as AD, Parkinson's, Huntington's, multiple sclerosis, and amyotrophic lateral sclerosis. In certain embodiments flavonoids alone, or in a pharmaceutical preparation, are administered through the nasal olfactory route. In certain embodiments the flavonoid is apigenin and the neural disease is Alzheimer's. Targeted dosages may affect mitochondrial function in brain neural cells thus ameliorating a neural disease. Further embodiments additionally administer a macrocyclic peptide that inhibits mitochondrial fission. In additional embodiments, a porosome complex is administered.

It has become increasing clear that age and/or diseases contributing to a decline in metabolism (e.g., diabetes), play an important role in the progression of neurodegenerative diseases such as AD. Small molecules that overcome such metabolic decline, therefore, serve as a therapeutic drug to treat AD.

Studies report that the flavonoid apigenin promotes mitochondrial biogenesis by activating the peroxisome proliferator-activated receptor Gamma Co-activator 1-alpha (PGC-1a), prevents neurodegeneration in rat hippocampus, and improves spatial working memory in rats. Apigenin will be used either orally, or via the nasal rout either alone or in combination with peptides targeted toward restoring normal metabolic function by preventing mitochondrial fission, in treating AD and other neurodegenerative diseases.

The unique anatomical and physiological features make it very effective to deliver drugs to the brain through nasal olfactory route, rather than via the systemic circulation. This way, the nasal cavity enables drugs to directly reach the brain, bypassing the blood-brain barrier. The nasal route of drug administration to the brain additionally overcomes metabolism when administered orally, decreases the amount of drug required, and minimizes the compliances associated with injectables into the systemic circulation, providing high pharmacological activity of drugs at lower dosages and with shorter half-lives.

Compelling evidence from basic research and clinical studies indicates that mitochondrial dysfunction is an early prominent event in AD neuropathology including Aβ plaque and neurofibrillary tangles (NFTs) and plays important roles in the pathogenesis of AD. Patients with AD are known to display diminished glucose utilization, abnormal brain energetics, mitochondrial DNA (mtDNA) lesion, and reduced activity of certain enzymes associated with mitochondrial respiratory chain complexes. Decreased ATP and respiratory activity, elevated mitochondrial reactive oxygen species (ROS), enhanced mitochondrial depolarization, and increased mtDNA depletion were also documented in various AD rodent models before disease onset, suggesting that mitochondrial dysfunction is an early event during AD progression. A direct interaction between Aβ and mitochondria is a relevant component of AD pathology. Aβ progressively accumulates within mitochondria in the brains of AD patients and rodent AD animals. The key components of the amyloid precursor protein (APP) processing and the β-amyloid producing γ-secretase complex, are highly enriched in the mitochondria, which results in local production of Aβ in the mitochondria. As a consequence, accumulation of Aβ in mitochondria disrupts mitochondrial bio-energetic activity, induces mitochondrial genome instability, increases ROS, and reduces protein degradation capacity, leading to programmed cell death, synaptic defects, and disease-associated pathology. In addition, localization of Tau within mitochondria or its association with the mitochondrial outer membrane has been documented. Tau positive neurons show marked mitochondrial loss. Pathological Tau has been reported to impair mitochondrial trafficking, mitochondrial dynamics and mitophagy. Thus, mitochondria might be an important route through which tau leads to the degeneration and death of neuronal cells. Eliminating damaged mitochondria altogether might be of importance to reduce AD pathology and related cognitive deficiency.

ATAD3A (ATPase family AAA-domain containing protein 3A) is a nuclear-encoded mitochondrial protein belonging to a family of AAA-ATPase proteins specific to multicellular eukaryotes. ATAD3A is a component of the mitochondrial nucleoid complex, which is required for mtDNA nucleoid maintenance. ATAD3A is structurally unique; its C-terminus, which contains the conserved ATPase, is located in the mitochondrial matrix, whereas its N-terminus has been found to be exposed to the cytosol and associate with the mitochondrial outer membrane. Remarkably, ATAD3A contains multiple transmembrane (TM) domains that allow it to traverse both inner-(IMM) and outer-mitochondrial membranes (OMM), at the IMM-OMM contact sites. ATAD3A has been found to regulate mitochondrial morphology and control cholesterol channeling for steroidogenesis at mitochondrial contact sites. Thus, ATAD3A, by straddling the two mitochondrial membranes, simultaneously regulates mitochondrial membrane integrity and mtDNA nucleoid organization.

The expression of mutant ATAD3A in Drosophila causes severe mitochondrial fragmentation, aberrant cristae, and increased mitophagy in both neurons and muscle, leading to early lethality. Although, global ATAD3A knockout (KO) is embryonic lethal, selective ATAD3A KO in mouse causes mitochondrial fragmentation and mitochondrial bioenergetics failure, resulting in cell death and tissue damage. ATAD3A mutation is also associated with axonal neuropathy and spastic paraplegia. Therefore, proper function of ATAD3A is crucial for mitochondrial activity, integrity and cellular survival. In AD, ATAD3A undergoes oligomerization, resulting in Drp1-mediated mitochondrial fragmentation, leading to neurodegeneration. Heterozygous knockdown of ATAD3A in AD transgenic mice abolishes ATAD3A oligomerization, reduces amyloid accumulation and neuroinflammation, and improves long-term memory.

Patients carrying a recently identified ATAD3A mutant show neurodegenerative conditions associated with developmental delay, axonal neuropathy, and spastic paraplegia. These data further support the idea that aberrant ATAD3A oligomerization is a key pathogenic factor to induce AD neuropathology and further validates ATAD3A as a drug target. Thus, embodiments of the disclosure deliver ATAD3A affecting peptides as part of a treatment for one or more neurological disorders. In certain embodiments, the ATAD3A affecting peptides are macrocyclic DA1 peptides that target ATAD3A oligomers.

Porosome organelles, or complexes, are cup-shaped supramolecular lipoprotein structures located at the cell plasma membrane. They are the sites at which secretory vesicles inside the cell transiently dock, fuse, and secret their contents outside the cell. They are the universal secretory machinery of the cell. The porosome structure includes multiple proteins with their associated ligands, chaperones, and other affiliated molecules such as lipids. Although it is classically understood that there are diseases caused by mutations/malformations in the structure of a single protein, it is only recently starting to be understood that malfunction and malformations of larger structures, such as the porosome, contribute to diseases.

Porosomes enable communication (language) between cells in the body by secreting chemical messages such as neurotransmitters from nerve cells or hormones from endocrine cells such as insulin from B-cells of the endocrine pancreas. These chemical messages are stored in secretory vesicles within the cell. The porosome secretory machinery, composed of 30+ proteins, provides instructions to secretory vesicles to appropriately dock at the porosome base, fuse, swell and release measured amounts of intra-vesicular contents to the outside. None of the individual components of the porosome are unique to it. Rather, the combination of 30 or more proteins together in the proper conformation within the complex, provides function to the structure. Thus, envisioned embodiments of this disclosure entail correcting cellular function by targeting a specific multimer, such as the porosome complex, without altering the activity of other cellular complexes, hence other cellular processes that possess one or more of the individual components of the targeted structure.

Typical porosome structures range in size from 15 nm in neurons to 100-180 nm in endocrine and exocrine cells. Porosomes are composed of about 30-40 proteins, with the porosome composition depending on cell type. Porosome-mediated secretion across the cell plasma membrane is a fundamental process through which cells communicate with their environment and exchange information. In a multicellular context, porosome secretion enables cell communities to communicate and maintain homeostasis and, thus, sustain life. Porosomes are present in all secretory cells, from the digestive enzyme-secreting pancreatic acinar cells to the hormone-releasing growth hormone and insulin-secreting cells, mast cells, chromaffin cells, hair cells of the inner ear, and in neurons secreting neurotransmitters. Porosomes have been immunoisolated from a number of cells including the insulin-secreting beta cells of the exocrine pancreas, cells of the human airways epithelia and neurons, biochemically characterized, and functionally reconstituted into artificial lipid membranes. A large body of evidence has accumulated on the role of porosome-associated proteins on cell secretion and secretory defects, including in neurotransmission and neurological disorders. Thus, defects in cell secretion stemming from porosome, or porosome component, malfunction are implicated to underpin numerous disease mechanisms, including those in many neurological disorders.

Tables containing lists of porosome proteins and protein-protein interactions are known and widely available to those of skill in the art. As used herein, a “porosome protein” is one that is a part, either singly or in multiple copies, of the overall porosome structure. Those of skill in the art can readily appreciate that porosomes associated with different cell/tissue types may have various proteins that compose them. As used herein, a “porosome-associated protein” is one that is found to interact with a porosome protein, or to interact with a porosome structure.

In an embodiment of the disclosure an entire functional porosome can be reconstituted into a targeted tissue such as, for instance, in the neural cells of a patient experiencing a neurological disease. In some embodiments, for the reconstitution, a porosome from pig or human sources is extracted and put in a human cell. In still other embodiments, the nanoscale porosome complex for reconstitution is obtained from CALU 3 or human neural cells to address the neural disease. Reconstitution therapy involves reconstituting or introducing a normal functional porosome complex at the cell plasma membrane of the neurons in patients experiencing a neurological disorder. Without subscribing to, or being bound by, a particular theory, reconstitution of a prosome complex coupled with a peptide inhibitor of mitochondrial fission, provided and/or dosed in sufficient amounts are sufficient to treat, ameliorate symptoms, or otherwise alter the course of a neurological disorder. It is known to practitioners that porosomes reconstituted into live cells are stable and functional.

In AD, the proteins 2,3-cyclic nucleotide phosphodiesterase (CNPase) and the heat shock protein 70 (HSP70) are implicated as playing a role in disease pathology. The levels of CNPase and HSP70, both present in the neuronal porosome complex are found to increase, while the levels of porosome-associated dihydropyrimidinase-related protein-2 (DRP-2) is decreased. Similarly, porosome proteins SNAP-25 and synaptophysin are significantly reduced in neurons of patients with AD.

Decreased levels of CNPase have also been reported in the frontal and temporal cortex of patients with AD and Down syndrome. Low CNPase levels have also been detected in the anterior frontal cortex in schizophrenic patients. Additionally, an allele that is associated with low levels of CNPase is also reported to be linked to Schizophrenia.

Examples of neuronal porosome proteins can include: Tubulin beta, myosin 7b, spectrin, Creatine kinase, Dystrophin, Langerin, GTPase activating protein (GAP), Intersectin 1 isoform (ITSN-1), Actin, cytoplasmic 1, Sodium/potassium-transporting ATPase subunit alpha-3, Plasma membrane calcium-transporting ATPase 1, Plasma membrane calcium-transporting ATPase 2, Brain acid soluble protein 1, Adenylyl cyclase-associated protein 1, 2′,3′-Cyclic-nucleotide 3′-phosphodiesterase, Dihydropyrimidinase-related protein 2, Dihydropyrimidinase-related protein 3, Dihydropyrimidinase-related protein 5, Glutamine synthetase, Guanine nucleotide-binding protein G (o) subunit alpha, Neural cell adhesion molecule 1, Vesicle-fusing ATPase, Ras-related protein Rab-3A, Reticulon-3, Reticulon-4, Synaptosomal-associated protein 25, Syntaxin-1A, Syntaxin-1B, Syntaxin-binding protein 1, Synapsin-2, Synaptophysin, Synaptotagmin-1, Tubulin alpha-1A chain, Vesicle-associated membrane protein 1, Vesicle-associated membrane protein 2, V-type proton ATPase subunit B, brain isoform. Embodiments of the invention can include one or more identified small molecules that directly act upon one or more of the above proteins to affect neuronal porosome structure and/or function.

Small molecule inhibitors & stimulators of porosome phosphodiesterase such as Vinpocetine, BAY 60-7550, Rolipram, Etazolate, Sildenafil, S14, VP1.15, PF-04447943, Papaverine, and the small molecule inhibitors Apoptozole, VER155008, JG98, HA15 and YUM70 of HSP70, and small molecule activator ML346 for HSP70, all may be used to treat neuronal diseases especially Alzheimer's.

In some embodiments, the porosome complex is isolated from cells or cell lysate obtained from a mammalian cell. In some instances, the mammalian cell is an epithelial cell, connective tissue cell, hormone secreting cell, a nerve cell, a skeletal muscle cell, a blood cell, or an immune system cell. In certain embodiments the cell may one sampled from a subject and subsequently proliferated.

Exemplary mammalian cells include, but are not limited to, 293A cell line, 293 FT cell line, 293F cells, 293 H cells, HEK 293 cells, CHO DG44 cells, CHO-S cells, CHO-K1 cells, Expi293F™ cells, Flp-In™ T-REX™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REX™ Jurkat cell line, Per.C6 cells, T-REX™-293 cell line, T-REx™-CHO cell line, T-REX™-HeLa cell line, NC-HIMT cell line, and PC12 cell line.

In some instances, the porosome containing cell sample or cell lysate sample is obtained from cells of a tumor cell line. In some instances, the cell sample or cell lysate sample is obtained from cells of a solid tumor cell line. In some instances, the solid tumor cell line is a sarcoma cell line. In some instances, the solid tumor cell line is a carcinoma cell line. In some embodiments, the sarcoma cell line is obtained from a cell line of alveolar rhabdomyosarcoma, alveolar soft part sarcoma, ameloblastoma, angiosarcoma, chondrosarcoma, chordoma, clear cell sarcoma of soft tissue, dedifferentiated liposarcoma, desmoid, desmoplastic small round cell tumor, embryonal rhabdomyosarcoma, epithelioid fibrosarcoma, epithelioid hemangioendothelioma, epithelioid sarcoma, esthesioneuroblastoma, Ewing sarcoma, extrarenal rhabdoid tumor, extraskeletal myxoid chondrosarcoma, extraskeletal osteosarcoma, fibrosarcoma, giant cell tumor, hemangiopericytoma, infantile fibrosarcoma, inflammatory myofibroblastic tumor, Kaposi sarcoma, leiomyosarcoma of bone, liposarcoma, liposarcoma of bone, malignant fibrous histiocytoma (MFH), malignant fibrous histiocytoma (MFH) of bone, malignant mesenchymoma, malignant peripheral nerve sheath tumor, mesenchymal chondrosarcoma, myxofibrosarcoma, myxoid liposarcoma, myxoinflammatory fibroblastic sarcoma, neoplasms with perivascular epitheioid cell differentiation, osteosarcoma, parosteal osteosarcoma, neoplasm with perivascular epitheioid cell differentiation, periosteal osteosarcoma, pleomorphic liposarcoma, pleomorphic rhabdomyosarcoma, PNET/extraskeletal Ewing tumor, rhabdomyosarcoma, round cell liposarcoma, small cell osteosarcoma, solitary fibrous tumor, synovial sarcoma, telangiectatic osteosarcoma.

In some embodiments, the carcinoma cell line is obtained from a cell line of adenocarcinoma, squamous cell carcinoma, adenosquamous carcinoma, anaplastic carcinoma, large cell carcinoma, small cell carcinoma, anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, or vulvar cancer.

In some instances, the porosome containing cell sample or cell lysate sample is obtained from cells of a hematologic malignant cell line. In some instances, the hematologic malignant cell line is a T-cell cell line. In some instances, B-cell cell line. In some instances, the hematologic malignant cell line is obtained from a T-cell cell line of: peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas.

In some instances, the hematologic malignant cell line is obtained from a B-cell cell line of: acute lymphoblastic leukemia (ALL), acute myelogenous leukemia (AML), chronic myelogenous leukemia (CML), acute monocytic leukemia (AMOL), chronic lymphocytic leukemia (CLL), high-risk chronic lymphocytic leukemia (CLL), small lymphocytic lymphoma (SLL), high-risk small lymphocytic lymphoma (SLL), follicular lymphoma (FL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some embodiments, the porosome containing cell sample or cell lysate sample is obtained from a tumor cell line. Exemplary tumor cell line includes, but is not limited to, 600MPE, AU565, BT-20, BT-474, BT-483, BT-549, Evsa-T, Hs578T, MCF-7, MDA-MB-231, SkBr3, T-47D, HeLa, DU145, PC3, LNCaP, A549, H1299, NCI-H460, A2780, SKOV-3/Luc, Neuro2a, RKO, RKO-AS45-1, HT-29, SW1417, SW948, DLD-1, SW480, Capan-1, MC/9, B72.3, B25.2, B6.2, B38.1, DMS 153, SU.86.86, SNU-182, SNU-423, SNU-449, SNU-475, SNU-387, Hs 817.T, LMH, LMH/2A, SNU-398, PLHC-1, HepG2/SF, OCI-Ly1, OCI-Ly2, OCI-Ly3, OCI-Ly4, OCI-Ly6, OCI-Ly7, OCI-Ly10, OCI-Ly18, OCI-Ly19, U2932, DB, HBL-1, RIVA, SUDHL2, TMD8, MEC1, MEC2, 8E5, CCRF-CEM, MOLT-3, TALL-104, AML-193, THP-1, BDCM, HL-60, Jurkat, RPMI 8226, MOLT-4, RS4, K-562, KASUMI-1, Daudi, GA-10, Raji, JcKo-1, NK-92, and Mino.

In some embodiments, the porosome containing cell sample or cell lysate sample is from any tissue or fluid from an individual. Samples include, but are not limited to, tissue (e.g. connective tissue, muscle tissue, nervous tissue, or epithelial tissue), whole blood, dissociated bone marrow, bone marrow aspirate, pleural fluid, peritoneal fluid, central spinal fluid, abdominal fluid, pancreatic fluid, cerebrospinal fluid, brain fluid, ascites, pericardial fluid, urine, saliva, bronchial lavage, sweat, tears, car flow, sputum, hydrocele fluid, semen, vaginal flow, milk, amniotic fluid, and secretions of respiratory, intestinal or genitourinary tract. In some embodiments, the cell sample or cell lysate sample is a tissue sample, such as a sample obtained from a biopsy or a tumor tissue sample. In some embodiments, the cell sample or cell lysate sample is a blood serum sample. In some embodiments, the cell sample or cell lysate sample is a blood cell sample containing one or more peripheral blood mononuclear cells (PBMCs). In some embodiments, the cell sample or cell lysate sample contains one or more circulating tumor cells (CTCs). In some embodiments, the cell sample or cell lysate sample contains one or more disseminated tumor cells (DTC, e.g., in a bone marrow aspirate sample).

In some embodiments, the porosome containing cell sample or cell lysate sample is obtained from an individual by any suitable means of obtaining the sample using well-known and routine clinical methods. Procedures for obtaining tissue samples from an individual are well known. For example, procedures for drawing and processing tissue sample such as from a needle aspiration biopsy is well-known and is employed to obtain a sample for use in the methods provided. Typically, for collection of such a tissue sample, a thin hollow needle is inserted into a mass such as a tumor mass for sampling of cells that, after being stained, would be examined under a microscope.

Neurotransmitter Release: As above mentioned, porosomes are secretory portals at the cell plasma membrane where secretory vesicles transiently dock and fuse to expel a precise amount of intra-vesicular contents from the cell during secretion. In neurons, porosomes are 15 nm cup-shaped lipoprotein structures at the presynaptic membrane, composed of nearly 40 proteins. A number of porosome proteins have previously been implicated in neurotransmission and neurological disorders, attesting to the crosstalk between porosome proteins and their coordinated involvement in release of neurotransmitter at the synapse. In AD, levels of porosome proteins CNPase (2,3-cyclic nucleotide phosphodiesterase) and the heat shock protein 70 (HSP70) are found to increase, while the levels of dihydropyrimidinase-related protein-2 (DRP-2) decrease. Decreased levels of CNPase have been observed in the frontal and temporal cortex of patients with AD. Similarly, porosome proteins SNAP-25 and synaptophysin are significantly reduced in neurons of patients with AD. Mice that are SNAP-25 (+/−) show disabled learning and memory, and exhibit epileptic like seizures. These results support that, alteration of one porosome protein impacts others within the complex, resulting in impaired porosome-mediated secretion. This is similar to recent studies on human bronchial epithelial (HBE) cells showing that HBE cells with ΔF508 cystic fibrosis transmembrane conductance regulator (CFTR) mutation, affects nearly a dozen porosome proteins including CFTR within the porosome complex. Therefore, the reprogramming of the porosome secretory machinery into the cell plasma membrane of Cystic Fibrosis (CF) cells, was able to rescue from CF. Thus, embodiments of the disclosure herein show that reconstitution of the 15 nm normal neuronal porosome complex in AD neurons overcome secretory defects in neurotransmitter release.

Neuronal Energy Metabolism: It is widely accepted that mitochondrial production of reactive oxygen species (ROS) contributes to the detrimental alterations in the etiology and/or progression of many pathological conditions, including brain neurodegeneration. Studies report that flavonoids can protect cells from different insults that lead to mitochondria-mediated cell death, and epidemiological data further show that some of these compounds attenuate the progression of diseases associated with oxidative stress and mitochondrial dysfunction. Flavonoids are low molecular weight phenolic compounds, displaying significant ROS scavenging capability, including other cellular antioxidant effects, hence ideal for the protection of brain neurons from buildup of free radicals generated by the mitochondria leading to mitochondrial fission in AD.

While various embodiments of the present disclosure are described herein, it will be understood by those skilled in the art that such embodiments are provided by way of example only. It will be understood by those skilled in the art that numerous modifications and changes to, and variations and equivalent substitutions of, the embodiments described herein can be made without departing from the scope of the disclosure. It is understood that various alternatives to the embodiments described herein may be employed in practicing the disclosure, and modifications may be made to adapt a particular structure or material to the teachings of the disclosure. It is also understood that every embodiment of the disclosure may optionally be combined with any one or more of the other embodiments described herein which are consistent with that embodiment.

Where elements are presented in list format (e.g., in a Markush group), it is understood that each possible subgroup of the elements is also disclosed, and any one or more elements can be removed from the list or group.

It is also understood that, unless clearly indicated to the contrary, in any method described or claimed herein that includes more than one act or step, the order of the acts or steps of the method is not necessarily limited to the order in which the acts or steps of the method are recited, but the disclosure encompasses embodiments in which the order is so limited.

It is further understood that, in general, where an embodiment in the description or the claims is referred to as comprising one or more features, the disclosure also encompasses embodiments that consist of, or consist essentially of, such feature(s).

It is also understood that any embodiment of the disclosure, e.g., any embodiment found within the prior art, can be explicitly excluded from the claims, regardless of whether the specific exclusion is recited in the specification.

Headings are included herein for reference and to aid in locating certain sections. Headings are not intended to limit the scope of the embodiments and concepts described in the sections under those headings, and those embodiments and concepts may have applicability in other sections throughout the entire disclosure.

All patent literature and all non-patent literature cited herein are incorporated herein by reference in their entirety to the same extent as if each patent literature or non-patent literature were specifically and individually indicated to be incorporated herein by reference in its entirety.

Unless otherwise defined, all technical and scientific terms used herein have the same meaning as commonly understood by one of ordinary skill in the art to which this disclosure belongs.

Where a range of values is provided, it is understood that each intervening value between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding both of those included limits are also included in the disclosure.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The term “exemplary” as used herein means “serving as an example, instance or illustration”. Any embodiment or feature characterized herein as “exemplary” is not necessarily to be construed as preferred or advantageous over other embodiments or features.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of’ or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of’ and “consisting essentially of’ shall be closed or semi-closed transitional phrases, respectively.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, in certain methods described herein that include more than one step or act, the order of the steps or acts of the method is not necessarily limited to the order in which the steps or acts of the method are recited unless the context indicates otherwise.

The term “about” or “approximately” means an acceptable error for a particular value as determined by one of ordinary skill in the art, which depends in part on how the value is measured or determined. In certain embodiments, the term “about” or “approximately” means within one standard deviation. In some embodiments, when no particular margin of error (e.g., a standard deviation to a mean value given in a chart or table of data) is recited, the term “about” or “approximately” means that range which would encompass the recited value and the range which would be included by rounding up or down to the recited value as well, taking into account significant figures. In certain embodiments, the term “about” or “approximately” means within 10% or 5% of the specified value. Whenever the term “about” or “approximately” precedes the first numerical value in a series of two or more numerical values or in a series of two or more ranges of numerical values, the term “about” or “approximately” applies to each one of the numerical values in that series of numerical values or in that series of ranges of numerical values.

Whenever the term “at least” or “greater than” precedes the first numerical value in a series of two or more numerical values, the term “at least” or “greater than” applies to each one of the numerical values in that series of numerical values.

Whenever the term “no more than” or “less than” precedes the first numerical value in a series of two or more numerical values, the term “no more than” or “less than” applies to each one of the numerical values in that series of numerical values.

Apigenin (4′,5,7-trihydroxyflavone; CAS NO: 520-36-5), found in many plants, is a natural product belonging to the flavone class that is the aglycone of several naturally occurring glycosides. It is a yellow crystalline solid that has been used to dye wool.

Pharmaceutical preparations or compositions described or used herein may further comprise coloring or stabilizing agents, osmotic agents, antibacterial agents, or any other substances as long as such substances do not interfere with the function of the composition. The pharmaceutical compositions of the instant disclosure, can, for example, be formulated as a solution, suspension, or emulsion in association with a pharmaceutically acceptable parenteral vehicle. Examples of such vehicles are water, saline, Ringer's solution, dextrose solution, and 5% human albumen. Liposomes may also be used. The vehicle may contain additives that maintain isotonicity (e.g., sodium chloride or mannitol) and chemical stability (e.g., buffers and preservatives). It should be appreciated that endotoxin contamination should be kept at a safe level, for example, less than 0.5 ng mg⁻¹ protein. Moreover, for human administration, preparations should meet sterility, pyrogenicity, general safety and purity standards as required by the United States Food and Drug Administration Office of Biological Standards. The formulations may be sterilized by commonly used techniques such as filtration.

The phrase “pharmaceutically acceptable” refers to substances and compositions which do not produce an adverse, allergic, or otherwise untoward reaction when administered to an animal, or a human, as appropriate. A substance which caused or produced any of these adverse effects would be classified as “biologically harmful’ within the scope of the present disclosure. Pharmaceutically acceptable substances and compositions include, but are not limited to solvents, dispersion media, coatings, antibacterial and antifungal agents, isotonic and absorption delaying agents. Except where incompatible with the disclosure the use of any conventional ingredient is contemplated. Furthermore, supplementary active ingredients which serve some other pharmacologically expedient purpose can also be incorporated into the instant compositions without departing from the broader scope of the instant disclosure.

The effective dose and method of administration of a particular embodiment of the instant disclosure may vary based on the individual patient and stage of any present diseases (e.g., breast cancer, HIV, other co-morbidities), as well as other factors known to those of skill in the art. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio of toxic to therapeutic effects is the therapeutic index, and it can be expressed as the ratio, LD50/ED50. Pharmaceutical compositions that exhibit large therapeutic indices are preferred. The data obtained from cell culture assays and animal studies is used in formulating a range of dosage for human use. The dosage of such compounds lies preferably within a range of circulating concentrations that include the ED50 with little or no toxicity. The dosage varies within this range depending upon the dosage form employed, sensitivity of the patient, and the route of administration.

Toxicity and safe dosage levels may be determined through the determination of dose-limiting toxicities (DLTs) and the overall DLT-rate (e.g., such as in the context of a clinical trial). In certain embodiments compositions with a DLT rate less than 25% are considered safe.

Effectiveness of embodiments disclosed herein may further be evaluated through cohort studies and examination of the recurrence-free survival (RFS) rate at chosen time intervals.

An “effective amount” of an agent or therapeutic peptide is an amount sufficient to achieve a desired therapeutic or pharmacological effect, such as an amount that is capable of activating the growth of neurons. An effective amount of an agent as defined herein may vary according to factors such as the disease state, age, and weight of the subject, and the ability of the agent to elicit a desired response in the subject. Dosage regimens may be adjusted to provide the optimum therapeutic response. An effective amount is also one in which any toxic or detrimental effects of the active compound are outweighed by the therapeutically beneficial effects.

The exact dosage is chosen by an individual physician in view of a patient to be treated. Dosage and administration are adjusted to provide sufficient levels of embodiments of the instant disclosure to maintain the desired effect (e.g., inducement of an immune response against uveal melanoma). Additional factors that may be taken into account include the severity of any disease state, age, weight, and gender of the patient; diet, time and frequency of the administration, drug combination(s), reaction sensitivities, and tolerance/response to therapy. Short acting pharmaceutical compositions are administered daily whereas long-acting pharmaceutical compositions are administered every 2, 3 to 4 days, every week, or once every two weeks or more. Depending on half-life and clearance rate of the particular formulation, the pharmaceutical compositions of the instant disclosure may be administered once, twice, three, four, five, six, seven, eight, nine, ten or more times per day.

Normal dosage amounts may vary from approximately 1 to 100,000 micrograms, up to a total dose of about 10 grams, depending upon the route of administration. Desirable dosages include 250 μg, 500 μg, 1 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 1 g, 1.1 g, 1.2 g, 1.3 g, 1.4 g, 1.5 g, 1.6 g, 1.7 g, 1.8 g, 1.9 g, 2 g, 3 g, 4 g, 5 g, 6 g, 7 g, 8 g, 9 g, and 10 g.

More specifically, the dosage of peptide agents described herein is one that provides sufficient peptide agent to attain a desirable effect, including stimulation of the immune system to induce a treatment effect or effecting the functioning of cellular mitochondria. Accordingly, the dose of the peptide agent preferably produces a tissue or blood concentration of both about 1 to 800 μM. Preferable doses produces a tissue or blood concentration of greater than about 10 μM to about 500 μM. Preferable doses are, for example, the amount of peptide required to achieve a tissue or blood concentration or both of 10 μM, 15 μM, 20 μM, 25 μM, 30 μM, 35 μM, 40 μM, 45 μM, 50 UM, 55 μM, 60 μM, 65 μM, 70 μM, 75 μM, 80 M, 85 μM, 90 μM, 95 μM, 100 μM, 110 μM, 120 μM, 130 μM, 140 μM, 150 μM, 160 μM, 170 μM, 180 μM, 190 μM, 200 μM, 220 μM, 240 μM, 250 μM, 260 μM, 280 μM, 300 μM, 320 μM, 340 μM, 360 μM, 380 μM, 400 μM, 420 μM, 440 μM, 460 μM, 480 μM, and 500 μM. Although doses that produce a tissue concentration greater than 800 μM are not necessarily preferred, they are envisioned and can be used with some embodiments of the present disclosure. A constant or cyclic infusion of embodiments of the disclosure can be provided to maintain a stable concentration of the therapeutic agents.

The pharmacologically active compounds of this invention can be processed in accordance with conventional pharmaceutical practices to produce medicinal agents for administration to patients (e.g., mammals, including humans). The peptides with, or without, modification can be incorporated into a pharmaceutical composition. Further, the manufacture of pharmaceuticals or therapeutic agents that deliver the peptides or a nucleic acid sequence encoding a peptide by several routes is an embodiment.

The term “administering” or “administer” to a patient includes dispensing, delivering or applying an active compound in a pharmaceutical formulation to a subject by any suitable route for delivery of the active compound to the desired location in the subject (e.g., to thereby contact a desired cell, such as a desired neuron), including administration into the cerebrospinal fluid or across the blood-brain barrier, delivery by either the parenteral or oral route, intramuscular injection, subcutaneous or intradermal injection, intravenous injection, buccal administration, transdermal delivery and administration by the rectal, colonic, vaginal, intranasal or respiratory tract route. The agents may, for example, be administered to a comatose, anesthetized, or paralyzed subject via an intravenous injection or may be administered intravenously to a pregnant subject to stimulate axonal growth in a fetus. Specific routes of administration may include topical application (such as by eyedrops, creams or erodible formulations to be placed under the eyelid, intraocular injection into the aqueous or the vitreous humor, injection into the external layers of the eye, such as via subconjunctival injection or subtenon injection, parenteral administration or via oral routes.

As used herein the term “sequence” explicitly contemplates DNA, cDNA, RNA and resulting peptide chains encoded thereby in both sense and antisense directions. To know one is to know the others via the standard rules of complementarity and codon encoding as exemplified in standardized DNA, RNA, and amino acid codon tables.

A “peptide” in the context of the present disclosure is to be understood as meaning a polymer composed of amino acids, preferably the 20 proteinogenic L-amino acids, preferably of linear structure, which has up to 100 amino acids which are linked to one another via peptide bonds. According to the disclosure, the peptides of the disclosure have an amino acid sequence of 4 to 50 amino acids. In the context of this disclosure, the amino acids are given in a one-letter code, where, for example, C stands for cysteine, R for arginine, A for alanine and L for leucine. It is further understood that unless otherwise indicated, the amino acids in an amino acid sequence disclosed herein are linked via peptide bonds and, unless otherwise indicated, the sequence is listed in N to C-terminal orientation.

Peptides can be chemically synthesized in various embodiments and/or recombinantly produced using protein design. Short peptides can easily be prepared synthetically, for example via solid phase synthesis. Longer peptides and polypeptides, on the other hand, are often produced recombinantly in A host organism.

Typical acidic or negatively charged amino acids (depending on pH) are D and E.

The positively charged or basic amino acids (depending on the pH value) typically include R, K and H.

Amino acids such as G, A, C, I, L, M, F, V, P, S, T, W, Y, N and Q are typically uncharged, i.e., neutral, amino acids.

When reference is made herein to an “any” amino acid, what is commonly meant is one of the 20 naturally occurring proteinogenic amino acids, i.e. one of glycine (G), alanine (A), valine (V), leucine (L), isoleucine (I), Phenylalanine (F), Serine(S), Threonine (T), Proline (P), Methionine (M), Cysteine (C), Histidine (H), Lysine (K), Arginine (R), Glutamine (Q), asparagine (N), aspartic acid (D), glutamic acid (E), tyrosine (Y) and tryptophan (W). Unless otherwise stated, the amino acids are typically L-amino acids. In alternative embodiments, the peptide can also consist of D-amino acids, although it may be preferred that D- and L-amino acids do not occur at the same time within the peptides described herein. In various embodiments, any such amino acid includes all the aforementioned amino acids with the exception of proline, or in some embodiments also with the exception of proline and glycine.

The identity of nucleic acid or amino acid sequences is determined by sequence comparison. This sequence comparison is based on the BLAST algorithm established and commonly used in the prior art (cf. e.g., Altschul et al. (1990) Basic local alignment search tool, J. Mol. Biol., 215:403-410, and Altschul et al. (1997) Gapped BLAST and PSI-BLAST: a new generation of protein database search programs, Nucleic Acids Res., 25:3389-3402) and basically happens by similar sequences of nucleotides or amino acids in the nucleic acid or amino acid sequences be assigned. A tabular assignment of the relevant positions is called alignment. Another algorithm available in the art is the FASTA algorithm. Sequence comparisons (alignments), especially multiple sequence comparisons, are created using computer programs. For example, the Clustal series (see e.g., Chenna et al. (2003) Multiple sequence alignment with the Clustal series of programs, Nucleic Acid Res., 31:3497-3500), T-Coffee (see e.g., Notredame et al. (2000) T-Coffee: A novel method for multiple sequence alignments, J. Mol. Biol., 302:205-217) or programs based on these programs or algorithms. Sequence comparisons (alignments) are also possible using the computer program Vector NTI® Suite 10.3 (Invitrogen Corporation, 1600 Faraday Avenue, Carlsbad, California, USA) with the specified standard parameters, whose AlignX module for sequence comparisons is based on ClustalW, or Clone Manager 10 (Use of the BLOSUM 62 scoring matrix for sequence alignment at the amino acid level). Unless otherwise stated, sequence identity reported herein is determined using the BLAST algorithm.

Such a comparison also allows a statement to be made about the similarity of the compared sequences to one another. It is usually given as percent identity, i.e., the proportion of identical nucleotides or amino acid residues in the same positions or in positions corresponding to one another in an alignment. The broader concept of homology includes conserved amino acid exchanges in amino acid sequences, i.e., amino acids with similar chemical activity, since these usually exert similar chemical activities within the protein. Therefore, the similarity of the compared sequences can also be stated as percent homology or percent similarity. Identity and/or homology information can be made for entire polypeptides or genes or just for individual regions. Homologous or identical regions of different nucleic acid or amino acid sequences are therefore defined by matches in the sequences. Such areas often have identical functions. They can be small and contain only a few nucleotides or amino acids. Such small areas often perform essential functions for the overall activity of the protein. It can therefore make sense to relate sequence matches only to individual, possibly small areas. Unless otherwise stated, identity or homology information in the present application refers to the total length of the nucleic acid or amino acid sequence specified in each case.

The peptide or protein concentration can be determined using known methods, for example the BCA method (bicinchoninic acid; 2,2′-biquinolyl-4,4′-dicarboxylic acid) or the biuret method (Gornall et al., J. Biol. Chem., 1948, 177:751-766). Those skilled in the art of peptide and protein technology will be aware of a variety of suitable methods for determining peptide or protein concentration that can be used within the scope of this disclosure.

Peptides according to the disclosure can have amino acid changes, in particular amino acid substitutions, insertions, or deletions. Such peptides are further developed, for example, through targeted genetic modification, i.e., through mutagenesis processes, and optimized for specific purposes or with regard to special properties (e.g., in terms of their stability, binding, etc.).

For example, targeted mutations such as substitutions, insertions or deletions can be introduced into the known molecules in order to change certain properties, for example. For this purpose, in particular the surface charges and/or the isoelectric point of the molecules and thereby their interactions with a surface can be changed. For example, the net charge of the peptides can be changed in order to influence substrate binding. Alternatively, or additionally, one or more corresponding mutations can, for example, increase the stability or adsorption of the peptide. Advantageous properties of individual mutations, e.g., individual substitutions, can complement each other.

The term “conservative amino acid substitution” means the exchange (substitution) of an amino acid residue for another amino acid residue, whereby this exchange does not lead to a change in polarity or charge at the position of the exchanged amino acid, e.g., the exchange of a non-polar amino acid residue for another non-polar amino acid residue. Conservative amino acid substitutions within the scope of the disclosure include, for example: G=A=S, I=V=L=M, D=E, N=Q, K=R, Y=F, S=T, G=A=I=V=L=M=Y=F=W=P=S=T.

In preferred embodiments, the peptide according to the disclosure can also be modified. Preferred modifications can be, for example, coupling the peptide with certain other molecules or chemical groups, for example organic (macro) molecules, for example via a covalent bond or a linker/spacer via a suitable amino acid of the chain and/or N- and/or C-terminal.

All the aforementioned features and embodiments can be implemented individually or in any combination.

Furthermore, the peptide according to the disclosure can also be at least one subunit (module) of a larger peptide or polypeptide, where the polypeptide can comprise a multimer of the sequences described herein, for example 1 to 30 repeats, more preferably 2 to 15 repeats, particularly preferably 2 to 10 repeats, e.g., 2, 3, 4, 5 or 6 repeats of the peptide. The polypeptide may include or consist of such multimers. The term “polypeptide” in this context refers in particular to those peptides that comprise 100 or more amino acids. The term “larger peptides” preferably refers to peptides with at least 40 amino acids, unless otherwise described.

In various embodiments, the peptide is a peptide or polypeptide (multimer) comprising two or more of the peptides as described herein. In various embodiments, the two or more peptides can be connected to one another by at least one spacer, preferably the at least one spacer comprises or consists of 1 to 10 amino acid residues, in particular 2, 3 or 4 amino acid residues, preferably selected from the group consisting of G, P, I, A and S or combinations thereof, in particular GPI or GAS. In such embodiments, the individual peptides are optionally connected linearly to one another via peptide bonds, possibly also via a spacer.

The peptides described herein may have been chemically synthesized in various embodiments and/or recombinantly produced using protein design. Nowadays, short peptides can easily be prepared synthetically, for example using solid-phase synthesis such as Merrifield's solid-phase synthesis. Longer peptides and polypeptides, on the other hand, are often produced recombinantly in the host organism, e.g., in bacteria or yeast.

It is preferred to produce the peptides and/or peptide conjugates according to the disclosure using recombinant processes. This includes all genetic engineering or microbiological processes that are based on the genes for the peptides of interest being introduced into a host organism suitable for production and transcribed and translated by it (summarized in the context of this disclosure as biotechnological processes).

The peptides and/or peptide conjugates according to the disclosure may be produced as polypeptides (multimers) and subsequently cleaved into the functional peptides and/or peptide conjugates. Very particularly preferred multimers have 1 to 30 peptide units (each according to the disclosure), each of which is separated from one another by spacers of 1 to 10 amino acids long (e.g., 1, 2, 3 or 4 amino acids). Alternatively, the spacers can also be or include interfaces for specific proteases/peptidases, in particular endopeptidases, or can form such an interface together with parts of the peptide.

Using methods that are generally known today, such as chemical synthesis or the polymerase chain reaction (PCR) in conjunction with standard molecular biological and/or protein chemical methods, it is possible for a person skilled in the art to identify the corresponding nucleic acids and even complete genes using known DNA and/or amino acid sequences to produce. Such methods are, for example, from Sambrook, J., Fritsch, E. F. and Maniatis, T. 2001. Molecular cloning: a laboratory manual, 3rd Edition Cold Spring Laboratory Press. known.

As used herein the terms “cyclic” and “macrocyclic” peptides are largely interchangeable. In general, “cyclic” peptides are smaller (˜12-20 amino acids) and form only one loop whereas “macrocyclic” peptides are larger (˜16-60 amino acids give or take) and form at least one loop in addition to potentially containing one more side chain modifiers and/or artificial amino acid substitutions. Macrocyclic peptides may have many structural forms or methods of creation. Example macrocyclic peptides can include: Multiple, site-selective disulfide bridges; (Cys-Cys, Pen-Cys, and Pen-Pen); Amide bond cyclizations (lactam); Head-to-tail, head-to-sidechain, sidechain-to-tail, sidechain-to-sidechain; Backbone-to-backbone, backbone-to-sidechain, backbone-to-head, and backbone-to-tail; Thioether bridges; hydrocarbon-stapled peptides; and, copper-catalyzed azide-alkyne cycloaddition (Click Chemistry).

In embodiments, peptides and/or peptide conjugates described herein are produced using biotechnological processes as described above.

The term “expression” refers to the process by which nucleic acid is translated into peptides or is transcribed into RNA, which, for example, can be translated into peptides, polypeptides, or proteins. If the nucleic acid is derived from genomic DNA, expression may, if an appropriate eukaryotic host cell or organism is selected, include splicing of the mRNA. For heterologous nucleic acid to be expressed in a host cell, it must initially be delivered into the cell and then, once in the cell, ultimately reside in the nucleus.

The terms “derived from” or “based on” in reference to peptide, amino acid, and nucleic acid sequences are viewed as standard terms of source attribution that may then be varied. For example, an RNA sequence may be derived from a DNA sequence and vice versa. Likewise, a peptide or amino acid sequence may be provided and a subsequent sequence based on that reference sequence created. Thus, to know that source or reference sequence is to have the basic element that may then be varied.

The term “heterologous nucleic acid sequence” is typically DNA that encodes RNA and proteins that are not normally produced in vivo by the cell in which it is expressed or that mediates or encodes mediators that alter expression of endogenous DNA by affecting transcription, translation, or other regulatable biochemical processes. A heterologous nucleic acid sequence may also be referred to as foreign DNA. Any DNA that one of skill in the art would recognize or consider as heterologous or foreign to the cell in which it is expressed is herein encompassed by heterologous DNA. Examples of heterologous DNA include, but are not limited to, DNA that encodes traceable marker proteins, such as a protein that confers drug resistance, DNA that encodes therapeutically effective substances, such as anti-cancer agents, enzymes and hormones, and DNA that encodes other types of proteins, such as antibodies. Antibodies that are encoded by heterologous DNA may be secreted or expressed on the surface of the cell in which the heterologous DNA has been introduced.

The terms “homology” and “identity” are used synonymously throughout and refer to sequence similarity between two peptides or between two nucleic acid molecules. Homology can be determined by comparing a position in each sequence, which may be aligned for purposes of comparison. When a position in the compared sequence is occupied by the same base or amino acid, then the molecules are homologous or identical at that position. A degree of homology or identity between sequences is a function of the number of matching or homologous positions shared by the sequences.

The term “nanoemulsion” (sometimes known as a “miniemulsion” by those of skill in the art) is a heterogeneous formulation of two different immiscible liquids (e.g., oil and water), often stabilized by surface-active agents (e.g., surfactants) to produce droplets within the nano-range (20-200 nm). Pharmaceutical nanoemulsions can be administered by SC, IM, intravenous, nasal, and mucosal routes.

The term “patient” or “subject” or “animal” or “host” refers to any mammal. The subject may be a human; but can also be a mammal in need of veterinary treatment, e.g., domestic animals (e.g., dogs, cats, and the like), farm animals (e.g., cows, sheep, fowl, pigs, horses, and the like) and laboratory animals (e.g., rats, mice, guinea pigs, and the like).

The terms “prevent” or “preventing” refer to reducing the frequency or severity of a disease or condition. The term does not require an absolute preclusion of the disease or condition. Rather, this term includes decreasing the chance for disease occurrence.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The agents, compounds, compositions, antibodies, etc. used in the methods described herein are considered to be purified and/or isolated prior to their use. Purified materials are typically “substantially pure”, meaning that a nucleic acid, polypeptide or fragment thereof, or other molecule has been separated from the components that naturally accompany it. Typically, the polypeptide is substantially pure when it is at least 60%, 70%, 80%, 90%, 95%, or even 99%, by weight, free from the proteins and other organic molecules with which it is associated naturally. For example, a substantially pure polypeptide may be obtained by extraction from a natural source, by expression of a recombinant nucleic acid in a cell that does not normally express that protein, or by chemical synthesis. “Isolated materials” have been removed from their natural location and environment. In the case of an isolated or purified domain or protein fragment, the domain or fragment is substantially free from amino acid sequences that flank the protein in the naturally-occurring sequence. The term “isolated DNA” means DNA has been substantially freed of the genes that flank the given DNA in the naturally occurring genome. Thus, the term “isolated DNA” encompasses, for example, cDNA, cloned genomic DNA, and synthetic DNA.

The terms “portion”, “fragment”, “variant”, “derivative” and “analog”, when referring to a polypeptide include any polypeptide that retains at least some biological activity referred to herein (e.g., inhibition of an interaction such as binding). Polypeptides as described herein may include portion, fragment, variant, or derivative molecules without limitation, so long as the polypeptide still serves its function. Polypeptides or portions thereof of the present invention may include proteolytic fragments, deletion fragments, or fragments that more easily reach the site of action when delivered to an animal.

Embodiments described herein relate to methods of inhibiting ATAD3A oligomerization, ATAD3A activation, and/or Drp1 activation in cells (e.g., nerve cells) of subjects with neurodegenerative disorders associated with aberrant ATAD3A activation and particularly relates to methods of treating disorders and/or neurodegenerative disorders associated with aberrant ATAD3A activation in a subject.

ATAD3A is a nuclear-encoded mitochondrial protein that spans the inner and outer membranes with its two terminal domains located in the outer membrane and the matrix. ATAD3A regulates mitochondrial morphology and controls cholesterol trafficking at mitochondrial contact sites. Either overexpression or downregulation of ATAD3A results in mitochondrial fragmentation, suggesting a scaffold-like property on maintenance of mitochondrial morphology. Moreover, ATAD3A is a component of mitochondrial nucleoid complex, which implicates in mtDNA nucleoid maintenance. While global knockout of ATAD3A is embryonic lethal, selective loss of ATAD3A in mouse skeletal muscle disrupts mitochondrial ultrastructure and reduces the number of cristae junctions, which impairs mtDNA integrity. The expression of mutant ATAD3A in Drosophila causes severe mitochondrial fragmentation, aberrant cristae, and increased mitophagy in both motor neurons and muscle, leading to early lethality. Patients carrying an ATAD3A mutant show neurodegenerative conditions associated with axonal neuropathy, and spastic paraplegia. The proper function of ATAD3A is therefore critical for cell survival.

In embodiments as used herein DA1 refers to a synthetic linear peptide corresponding to a homologous region between Drp1 and ATAD3A as known in the art. The sequence of linear DA1 is known to be highly conserved between species. SEQ. ID. NO: 1 presents an example sequence of a DA1 peptide: EDKRKT. Those of skill in the art can appreciate that conservative amino acid substitutions may be made as necessary. Those of skill in the art can further recognize that in certain circumstances one or more additional peptides of different sequences may also correspond to a homologous region between Drp1 and ATAD3A, such variants are known and contemplated as within the scope of this disclosure.

Linear DA1, binds to ATAD3A and reduces ATAD3A oligomerization, which in turn decreases Drp1/ATAD3A binding occurring during AD progression. Linear DA1 treatment reduces mitochondrial bioenergetic defects and improves survival of neurons exposed to toxic Aβ in vitro. Linear DA1 penetrates the blood-brain-barrier and is nontoxic with minimal effects on the immune response. In vivo tests of linear DA1 compared to a control peptide in 5×FAD AD transgenic mice showed that mice treated with the control peptide exhibited enhanced ATAD3A oligomerization at 6 months of age, which was abolished by treatment with linear DA1. Immunohistochemistry of 6-month-old 5×FAD mice brains revealed a significant increase in the density and area covered by amyloid beta (Aβ). Linear DA1 treatment significantly reduced the amyloid load. Furthermore, linear DA1 decreased immunoreactivity of GFAP (astrocytes) and Iba1 (microglia), suggesting reduced neuroinflammation. Additionally, linear DA1 treatment from 2 to 8 months of age improved the long-term memory of 5×FAD mice. Linear DA1 treatment had no effect on the behavior of WT mice, further supporting the nontoxic nature of the linear peptide.

### Selected Abbreviations

Aβ amyloid beta
AD Alzheimer's Disease
ATAD3A ATPase family AAA-domain containing protein 3 A
BBB blood brain barrier
Cmax maximum concentration
CNS central nervous system
DA1 (EDKRKT)—synthetic peptide corresponding to the Drp1 region of homology with ATAD3A

As above mentioned, Alzheimer's disease (AD) is a neurodegenerative disorder characterized by memory loss leading to dementia. It is estimated that in the United States, nearly 6.2 million individuals are impacted by AD dementia, and over 50 million globally. AD is increasing at an alarming pace, projected to double by 2050. According to the World Alzheimer's Report, the total estimated annual worldwide health care costs for persons with AD has reached almost a trillion dollars. Secretory defects in neurotransmitter release and synaptic plasticity, synaptic loss, and mitochondrial dysfunction, have been recognized as early events in AD pathogenesis. Brain regions, especially the entorhinal, temporal and fronto-parietal cortex, the hippocampus and the subcortical nuclei are primarily affected. There is growing evidence that in AD, there is impaired neuronal metabolism due to an increase in free radicals and mitochondrial fission, leading to loss in ATP synthesis required for neurotransmitter release.

Embodiments of the present disclosure present the evolution of the linear DA1 peptide into one or more cyclic DA1 analogues, having improved stability and efficacy. Also presented herein is the combinatorial use of porosome reconstitution therapy, a small flavonoid molecule and linear and cyclic DA1 synthetic peptides, in treating neuronal diseases (e.g., AD). Results in the combinatorial use of secretory (porosome) and metabolic (flavonoid and DA1 peptide) show usefulness for AD and additional neural disease therapy.

Unique anatomical and physiological features make it very effective to deliver drugs to the brain through the nasal olfactory route, rather than via systemic circulation. Administration of embodiments disclosed herein through the nasal cavity enables drugs to directly reach the brain, bypassing the blood-brain barrier. The nasal route of drug administration to the brain additionally overcomes metabolism of therapeutics which occurs when administered orally. Further advantages include a decrease in the amount of drug required, and minimization of noncompliance issues such as those associated with injectables into systemic circulation. Thus, embodiments of the disclosure favorably provide high pharmacological activity of drugs at lower dosages, and with shorter half-lives (e.g., the administration of drugs that would otherwise decrease in concentration or potency if administered systemically where the drug would be metabolized or removed from circulation).

Embodiments of the disclosure include a pharmaceutically acceptable preparation of apigenin. The preparation may be in a form suitable for administration through the nasal olfactory route. Such administration may be in the form of a suspended mist, powder, solution, nanoemulsion, or other forms such as are known in the art. Embodiments may further include at least one peptide. The at least one peptide may be associated with mitochondrial fission. Non-limiting examples of mitochondrial fission associated peptides can include: Drp 1 adaptor proteins, MiD49, MiD51, FISI, etc., The peptide may be linear or macrocyclic. In certain embodiments a combination of linear and macrocyclic peptides may be administered.

Further disclosed are a series of macrocyclic DA1 peptides targeting ATAD3A oligomers that improve CNS drug properties. The present disclosure underscores the potential of cyclic DA1 analogs as promising therapeutic candidates for AD, particularly by addressing mitochondrial dysfunction—a critical element in the pathogenesis of neurodegenerative disorders. The cyclic DA1 analogs herein disclosed advantageously overcome the limitations of linear DA1 peptides, such as metabolic instability and limited bioavailability. The disclosed analogs aimed to enhance metabolic stability while maintaining or improving on-target efficacy. The disclosed results indicate the successful creation of analogs with enhanced stability and efficacy. A particular embodiment, DA45, demonstrates superior pharmacokinetic profiles and brain permeability.

The broader implications of this disclosure suggest that cyclic DA1 analogs could be applicable to a range of neurodegenerative disorders beyond AD, particularly those characterized by mitochondrial dysfunction. The shared mechanistic role of ATAD3A oligomerization in both AD and HD, illustrates that targeting this pathway likely has therapeutic benefits across multiple conditions. The ability of cyclic DA1 analogs to modulate ATAD3A oligomerization and improve mitochondrial function indicates their further utility in treating diseases where mitochondrial integrity is compromised, such as Parkinson's disease (PD), amyotrophic lateral sclerosis (ALS), and certain forms of hereditary spastic paraplegia.

### Methods and Results—First Tests

Macrocyclic peptides are categorized into three groups based on their cyclization methods: sidechain-to-sidechain, head-to-sidechain, and head-to-tail (backbone) cycles. Embodiments disclosed herein enhance the efficacy and stability of a comparison linear DA1 peptide. The comparison DA1 linear peptide is structured as TAT-gly-gly-EDKRKT-NH2, where the TAT peptide carrier is linked to DA1 (EDKRKT) via a gly-gly linker, a configuration designed for cell permeability and biological activity.

In a general sense, to optimize the design, there was introduced a variety of cyclization linkers embodying techniques that ranged from disulfide bonds, which allow reversible cyclization, to more rigid bicyclic aromatic staples, which enforce a defined geometry and enhance structural stability. These cyclization strategies were employed with the goal of controlling the conformation of the peptide, thereby improving its target engagement and resistance to degradation.

In certain embodiments of the analogs disclosed herein, there was a substitution of regular L-amino acids with D-amino acids. D-amino acids are known for their resistance to proteolytic degradation, which can significantly enhance the stability of peptides in biological environments. This strategic modification was intended to further extend the half-life of the cyclic peptides while maintaining or enhancing their biological activity.

In total, fifty-four (54) cyclic DA1 analogs were synthesized. Some embodiments incorporated D-amino acid replacements, while others retained the standard L-amino acid structure. The diversity of the created analogs allowed the exploration and demonstration of a broad range of structural variations, each designed to optimize the stability, bioavailability, and on-target effects of the linear DA1 peptide. These analogs were subsequently subjected to a series of rigorous cell-based assays to evaluate their efficacy compared to the linear DA1 peptide. Table 1 presents the peptides prepared and tested.

TABLE 1
Cyclic DA1 peptides.

| | SEQ. | | | |
|---|---|---|---|---|
| Peptide | ID. | | | |
| No. | NO: | Custom ID | Peptide List | Commentary |
| Ref. | 1 | Linear DA1 | GRKKRRQRRR-GG-EDKRKT-NH2 | Original sequence |
| | 2 | WX-CWRU- | GRKKRRQRRR-GG-c(KEDKRKTE)- | Cyclic peptide, E and K for |
| | | 000001 | NH2 | cyclization |
| 2 | 3 | WX-CWRU- | GRKKRRQRRR-GG-c(KGEDKRKTGE)- | Enlarge the ring |
| | | 000002 | NH2 | |
| 3 | 4 | WX-CWRU- | GRKKRRQRRR-GG- | Enlarge the ring |
| | | 000003 | c(KGGEDKRKTGGE)-NH2 | |
| 4 | 5 | WX-CWRU- | GRKKRRQRRR-GG-edkrkt-NH2 | All D-amino acids |
| | | 000004 | | |
| 5 | 6 | WX-CWRU- | GRKKRRQRRR-GG-eDkRkT-NH2 | Half L-AA, half D-AA |
| | | 000005 | | |
| 6 | 7 | WX-CWRU- | GRKKRRQRRR-GG-EdKrKt-NH2 | Half L-AA, half D-AA |
| | | 000006 | | |
| 7 | 8 | WX-CWRU- | GRKKRRQRRR-GG-EDKrkt-NH2 | Half L-AA, half D-AA |
| | | 000007 | | |
| 8 | 9 | WX-CWRU- | GRKKRRQRRR-GG-edkRKT-NH2 | Half L-AA, half D-AA |
| | | 000008 | | |
| 9 | 10 | WX-CWRU- | GRKKRRQRRR-GG- | Add positive charge |
| | | 000009 | c(KRGEDKRKTGRE)-NH2 | |
| 10 | 11 | WX-CWRU- | GRKKRRQRRR-GG- | Add negative charge |
| | | 000010 | c(KEGEDKRKTGEE)-NH2 | |
| 11 | 12 | WX-CWRU- | GRKKRRQRRR-GG-c(KEDKRKTe)-NH2 | Combo |
| | | 000011 | | |
| 12 | 13 | WX-CWRU- | GRKKRRQRRR-GG-c(kgEDKRKTge)- | Combo |
| | | 000012 | NH2 | |
| 13 | 14 | WX-CWRU- | GRKKRRQRRR-GG-c(kggEDKRKTgge)- | Combo |
| | | 000013 | NH2 | |
| 14 | 15 | WX-CWRU- | GRKKRRQRRR-GG-c(KedkrktE)-NH2 | Combo |
| | | 000014 | | |
| 15 | 16 | WX-CWRU- | GRKKRRQRRR-GG-c(kedkrkte)-NH2 | Combo |
| | | 000015 | | |
| 16 | 17 | WX-CWRU- | GRKKRRQRRR-PEG2-EDKRKT-NH2 | Linker replacement |
| | | 000016 | | |
| 17 | 18 | WX-CWRU- | GRKKRRQRRR-PEG4-EDKRKT-NH2 | Linker replacement |
| | | 000017 | | |
| 18 | 19 | WX-CWRU- | GRKKRRQRRR-GG-c(CEDKRKTC)- | Cyclization by disulfide bond |
| | | 000018 | NH2 | |
| 19 | 20 | WX-CWRU- | GRKKRRQRRR-GG- | Enlarge the cycle |
| | | 000019 | c(CGEDKRKTGC)-NH2 | |
| 20 | 21 | WX-CWRU- | GRKKRRQRRR-GG-c(CedkrktC)-NH2 | Combo |
| | | 000020 | | |
| 21 | 22 | WX-CWRU- | GRKKRRQRRR-GG-c(CgedkrktgC)-NH2 | Combo |
| | | 000021 | | |
| 22 | 23 | WX-CWRU- | GRKKRRQRRR-GG-ADKRKT-NH2 | Replacement of Ala with one |
| | | 000022 | | aa |
| 23 | 24 | WX-CWRU- | GRKKRRQRRR-GG-EAKRKT-NH2 | Replacement of Ala with one |
| | | 000023 | | aa |
| 24 | 25 | WX-CWRU- | GRKKRRQRRR-GG-EDARKT-NH2 | Replacement of Ala with one |
| | | 000024 | | aa |
| 25 | 26 | WX-CWRU- | GRKKRRQRRR-GG-EDKAKT-NH2 | Replacement of Ala with one |
| | | 000025 | | aa |
| 26 | 27 | WX-CWRU- | GRKKRRQRRR-GG-EDKRAT-NH2 | Replacement of Ala with one |
| | | 000026 | | aa |
| 27 | 28 | WX-CWRU- | GRKKRRQRRR-GG-EDKRKA-NH2 | Replacement of Ala with one |
| | | 000027 | | aa |
| 28 | 29 | WX-CWRU- | GRKKRRQRRR-GG-S5EDKRKTR8- | Stapled peptide by RCM |
| | | 000028 | NH2 | |
| 29 | 30 | WX-CWRU- | GRKKRRQRRR-GG- | Stapled peptide by RCM |
| | | 000029 | c(S5GEDKRKTGR8)-NH2 | |
| 30 | 31 | WX-CWRU- | GRKKRRQRRR-βAβA-EDKRKT-NH2 | Linker replacement |
| | | 000030 | | |
| 31 | 32 | WX-CWRU- | GRKKRRQRRR-GG-c(KRGedkrktGRE)- | Combo |
| | | 000031 | NH2 | |
| 32 | 33 | WX-CWRU- | GRKKRRQRRR-GG-c(KEGedkrktGEE)- | Combo |
| | | 000032 | NH2 | |
| 33 | 34 | WX-CWRU- | GRKKRRQRRR-GG-c(KRedkrktRE)- | Combo |
| | | 000033 | NH2 | |
| 34 | 35 | WX-CWRU- | GRKKRRQRRR-GG-c(kredkrktre)-NH2 | Combo |
| | | 000034 | | |
| 35 | 36 | WX-CWRU- | GRKKRRQRRR-GG- | Add positive charge |
| | | 000035 | c(KKGEDKRKTGKE)-NH2 | |
| 36 | 37 | WX-CWRU- | GRKKRRQRRR-GG- | Add negative charge |
| | | 000036 | c(KDGEDKRKTGDE)-NH2 | |
| 37 | 38 | WX-CWRU- | GRKKRRQRRR-GG- | Combo |
| | | 000037 | c(kkgEDKRKTgke)-NH2 | |
| 38 | 39 | WX-CWRU- | GRKKRRQRRR-GG- | Combo |
| | | 000038 | c(kdgEDKRKTgde)-NH2 | |
| 39 | 40 | WX-CWRU- | GRKKRRQRRR-GG-EDhArgRKT-NH2 | Replacement of K with |
| | | 000039 | | homoarginine |
| 40 | 41 | WX-CWRU- | GRKKRRQRRR-GG-EDKRhArgT-NH2 | Replacement of K with |
| | | 000040 | | homoarginine |
| 41 | 42 | WX-CWRU- | GRKKRRQRRR-GG-EDhArgRhArgT- | Replacement of both K with |
| | | 000041 | NH2 | homoarginine |
| 42 | 43 | WX-CWRU- | grkkrrqrrr-GG-c(kEDKRKTe)-NH2 | D-TAT with Wuxi 11 |
| | | 000042 | | replacement |
| 43 | 44 | WX-CWRU- | grkkrrqrrr-GG-c(kggEDKRKTgge)- | D-TAT with Wuxi |
| | | 000043 | NH2 | 13replacement |
| 44 | 45 | WX-CWRU- | grkkrrqrrr-GG-c(KedkrktE)-NH2 | D-TAT with Wuxi 14 |
| | | 000044 | | replacement |
| 45 | 46 | WX-CWRU- | grkkrrqrrr-GG-c(kedkrkte)-NH2 | D-TAT with Wuxi 15 |
| | | 000045 | | replacement |
| 46 | 47 | WX-CWRU- | grkkrrqrrr-GG-c(KGGEDKRKTGGE)- | D-TAT with Wuxi 3 |
| | | 000046 | NH2 | replacement |
| 47 | 48 | WX-CWRU- | grkkrrqrrr-GG-c(KRGEDKRKTGRE)- | D-TAT with Wuxi 9 |
| | | 000047 | NH2 | replacement |
| 48 | 49 | WX-CWRU- | GRKKRRQRRR-GG- | Enlarge the ring further |
| | | 000048 | c(KRKGEDKRKTGGDE)-NH2 | (macrocyclic) |
| 49 | 50 | WX-CWRU- | GRKKRRQRRR-GG- | Enlarge the ring further |
| | | 000049 | c(KRKGEDKRKTGGRE)-NH2 | (macrocyclic) |
| 50 | 51 | WX-CWRU- | GRKKRRQRRR-GG- | Enlarge the ring further |
| | | 000050 | c(KRKTEDKRKTEDGE)-NH2 | (macrocyclic) |
| 51 | 52 | WX-CWRU- | GRKKRRQRRR-GG-c(CEDKRTC)NH2 | |
| | | 000051 | | |
| 52 | 53 | WX-CWRU- | GRKKRRQRRR-GG-c(cedkrtc)NH2 | |
| | | 000052 | | |
| 53 | 54 | WX-CWRU- | GRKKRRQRRR-GG-c(ckrgedkrtgrec)- | |
| | | 000053 | NH2 | |
| 54 | 55 | WX-CWRU- | GRKKRRQRRR-GG-c(ckedkrkteC)-NH2 | |
| | | 000054 | | |

As illustrated in the listing found in Table 1, some general principles guided the creation and testing of the cyclic peptides. To improve the stability of the created cyclic peptide and retain the integrity of the effective sequence EDKRKT, the main optimization strategies fall into the categories of:
1) cyclization of the original linear peptide to increase the structural rigidity of the peptide;
2) replacement of L-amino acids (upper case letters) with D-amino acids (lower case letters) to be less susceptible towards proteolytic enzymes;
3) combination of cyclization and D-transformation; and,
4) replacement of linker GG.

In terms of cyclization of the peptide from the linear version, the synthetic cyclic peptides were formed by the general processes comprising:
A) Amide bonding by adding glutamic acid and lysine to the C- and N-terminus of the effective sequence EDKRKT, respectively, to form the ring connected by their side chain.
B) Disulfide cyclic peptide was designed and created with the addition of 2 cysteines at the end of the sequence EDKRKT.
C) Stapled peptides, produced by adding amino acids S5 (S-2-(4′-pentenyl) alanine) and R8 (R-2-(7′-octenyl) alanine) to the ends of EDKRKT, respectively, forming the RCM cyclic peptide. Glycine was incorporated appropriately to expand the ring to ensure that the sequence EDKRKT can still be targeted effectively.

As above mentioned, there was substitution of D-amino acids for L-amino acids, given that most enzymes in humans target L-amino acids. D-substitution for TAT or/and EDKRKT sequences was taken to improve in vivo stability (Lowercase letters in Table 1 are used to designate D-form amino acids and uppercase letters are used for L-form amino acids.)

The combination of the above 2 strategies was performed for a synergic purpose of peptide stability enhancement (eg. for WX-CWRU 14-15, 20-21).

It was also explored whether the stability of the whole peptide could be improved by replacing the linker GG with PEG or β-alanine (eg. for WX-CWRU 16-17, 30).

In addition, positive and negative amino acids were incorporated to study if the stability is charge dependent, as well as introducing homoarginine to improve stability.

After screening fifty-four cyclic peptides (Table 1, screening below described), it was found that peptides #42, #44, #45, and #47 outperformed the linear peptide. Notably, #44 and #45 demonstrated metabolic stability. Both #44 and #45 align with the general principle outlined in category 3: a combination of cyclization and D-transformation.

#44 design (grkkrrrqrrr-GG-c (KedkrktE)-NH2): The cell-penetrating peptide TAT was substituted with all D-type amino acids and attached to the cyclic peptide via a GG linker, where the L-form amino acids glutamic acid and lysine were integrated into the C- and N-terminus of D-form EDKRKT for cyclic peptide formation.

#45 design (grkkrrrrrr-GG-c (kedkrkte)-NH2): The cell-penetrating peptide TAT was substituted with all D-type amino acids and attached to the all D-type cyclic peptide via a GG linker.

### Screening Cyclic DA1 in AD Neuronal Culture

An extensive screening was conducted of fifty-four (54) cyclic DA1 analogs (see Table 1) using a series of cell-based assays designed to evaluate their efficacy in modulating stress-induced ATAD3A oligomerization, mitigating mitochondrial oxidative stress, and improving cell viability in mouse hippocampal neuronal HT-22 cells exposed to toxic amyloid-beta (Aβ1-42) at a concentration of 5 μM. These assays included the use of the MitoSOX fluorescence probe to quantify mitochondrial oxidative stress and a viability assay to assess the protective effects of the analogs.

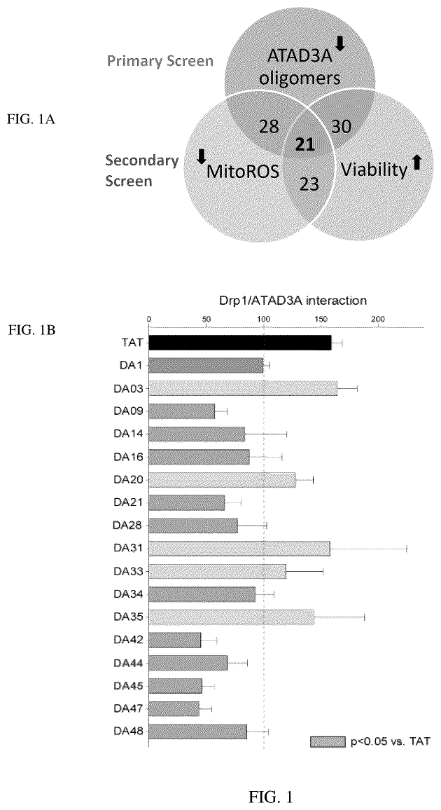

Seen in FIG. 1, the screening identified 21 cyclic DA1 analogs that matched or exceeded the efficacy of the linear DA1 peptide (1 μM) across all measured parameters (FIG. 1A). Specifically, the 21 analogs significantly inhibited the formation of ATAD3A oligomers, reduced mitochondrial oxidative stress, and improved cell viability in HT-22 cells under toxic Aβ conditions. The parental linear DA1 peptide was originally engineered to disrupt the protein-protein interaction between Drp1 and ATAD3A, thereby blocking the formation of ATAD3A oligomers. Remarkably, 11 of these 21 positive analogs demonstrated a robust ability to inhibit the Drp1-ATAD3A interaction under stress conditions, further validating their on-target efficacy and potential therapeutic utility.

### Measurement of Stability of Cyclic DA1 Peptide In Vitro

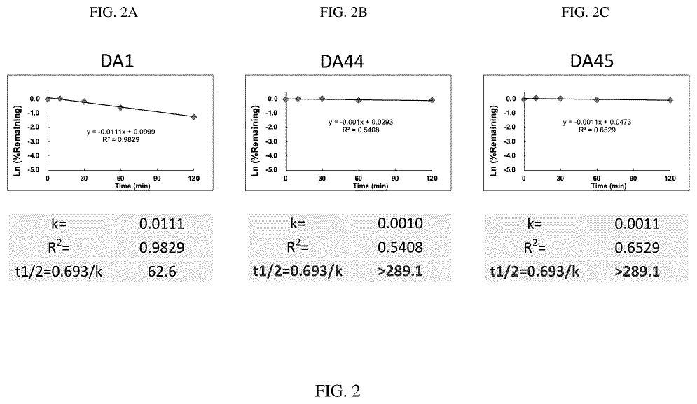

Evaluation of the top-performing analogs occurred following the efficacy screening. Seen in FIG. 1B, in vitro mouse plasma stability and hepatocyte stability assays were conducted on the eleven (11) final positive hits to determine their half-lives and overall stability. As. Seen in FIG. 2, among these analogs, DA44 (FIG. 2B) and DA45 (FIG. 2C) emerged as particularly promising candidates, exhibiting remarkable stability with half-lives exceeding 289 minutes in the mouse plasma stability assay. Additionally, the two analogs maintained impressive stability in hepatocyte assays, with half-lives surpassing 216 minutes (Table 2). These stability profiles were significantly superior to those of other positive analogs and the parental DA1 peptide (FIG. 2A).

The combination of enhanced efficacy in cell-based assays and superior metabolic stability position DA44 and DA45 as preferred embodiments of the disclosure. The presented findings underscore the success of embodiments of the disclosure; offering high stability and potent on-target effects; and, offering promising therapy for conditions involving ATAD3A oligomerization.

TABLE 2
In vitro metabolic stability of hepatocytes

| | | Mouse | | | | |
|---|---|---|---|---|---|---|
| | Cell Conc. | | | | CLint (hep) | CLint (hep) |
| Compound ID | (×10⁶ cells/mL) | R2 | k | T_{1/2}(min) | (μL/min/10⁶) | (mL/min/kg) |
| DA1 | 0.5 | NA | NA | <7.5 | >184.8 | >2195.9 |
| DA44 | | 0.3231 | −0.0032 | >216.8 | <6.4 | <75.9 |
| DA45 | | 0.0848 | −0.0012 | >216.8 | <6.4 | <75.9 |
| 7-Ethoxycoumarin | | 1.0000 | 0.2548 | 2.7 | 509.6 | 6054.6 |
| 7-Hydroxycoumarin | | 1.0000 | 0.2330 | 3.0 | 466.1 | 5536.7 |

### Efficacy of Top Cyclic DA1 Analogs in AD Neuronal Culture

Cyclic DA44 and DA45 were assessed for their efficacy in an AD neuronal culture model. The model was composed of mouse hippocampal neuronal HT-22 cells which were subjected to treatment with toxic amyloid-beta (Aβ) to induce cellular stress and mimic AD-like conditions. Following Aβ exposure, the cells were treated with cyclic DA44, DA45 or control peptide TAT, 1 μM each.

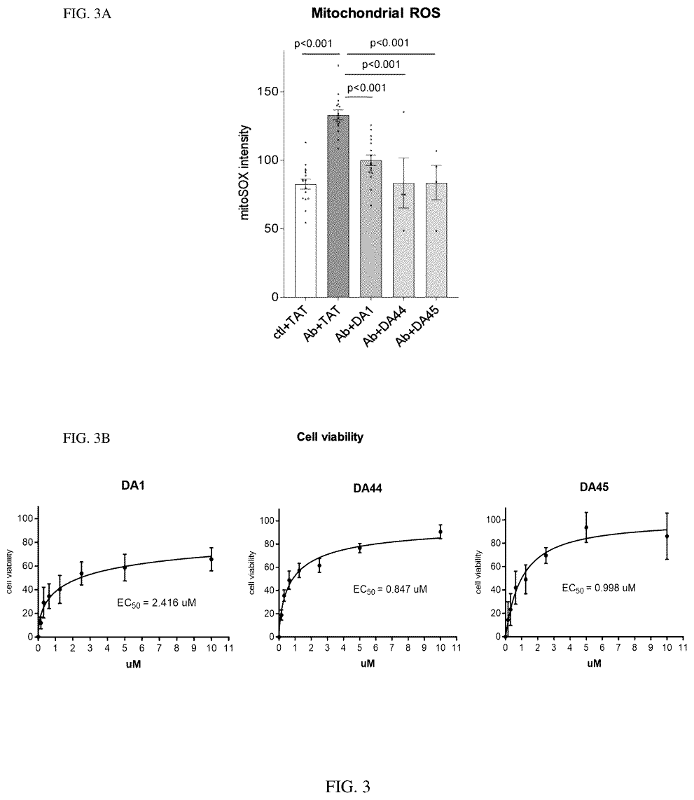

Test results revealed that treatment with cyclic DA44 and DA45 led to a significant reduction in mitochondrial reactive oxygen species (ROS) levels, indicating a protective effect against oxidative stress (FIG. 3A). Furthermore, dose-response studies demonstrated that cyclic DA44 and DA45 exhibit effective dose (ED50) values of approximately 0.847 μM and 0.998 μM, respectively, in improving cell viability. These data were lower than that of the control linear DA1 peptide, which exhibited an ED50 of approximately 2.416 UM under the same conditions (FIG. 3B). This suggests that the cyclic versions of DA1 are more potent in enhancing cell survival in the context of AD-like neurotoxicity.

Collectively, the data provide compelling evidence that cyclic DA44 and DA45 offer superior efficacy compared to the linear DA1 peptide in an AD cell culture model. The enhanced potency of these cyclic analogs further highlights the ability of the approach disclosed herein to advantageously provide therapeutic agents.

### Pharmacokinetic Profile of Cyclic DA45

The pharmacokinetic profile of cyclic DA45 (acetate) was investigated following a 10 mg/kg subcutaneous injection in wild-type mice. Pharmacokinetic analysis revealed that cyclic DA45 exhibited a half-life of approximately 31.4 hours in the brain and 4.6 hours in plasma, indicating a sustained presence in both compartments (Table 3). These half-lives suggest that cyclic DA45 has a favorable pharmacokinetic profile for prolonged action within the central nervous system (CNS).

TABLE 3
In vivo pharmacokinetics analysis of DA45. C57Black mice were subcutaneously
treated with DA45 acetate (10 mg/kg). The brain and plasma samples were
harvested at various time points after treatment. The concentration of
DA45 was then measured by LC/MS/MS. n = 3 mice/time point.

| PK Parameters of DA45 acetate | | PK Parameters of DA45 acetate | |
|---|---|---|---|
| | Subcutaneous treatment | | Subcutaneous treatment |
| | (10 mg/kg) | | (10 mg/kg) |
| PK Parameters | Mean Brain (ng/g) | PK Parameters | Plasma (ng/mL) |
| No. points used | 3 | No. points used | 3 |
| for T_{1/2} | | for T_{1/2} | |
| Cₘₐₓ (ng/g) | 154.0 | Cₘₐₓ (ng/g) | 8105.3 |
| Tₘₐₓ (h) | 9.0 | Tₘₐₓ (h) | 8.6 |
| T_{1/2} (h) | 31.4 | T_{1/2} (h) | 4.6 |
| Tₗₐₛₜ (h) | 32.0 | Tₗₐₛₜ (h) | 32.0 |
| AUC₀₋₄ (ng · h/g) | 136.9 | AUC₀₋₄ (ng · h/g) | 7718.4 |
| AUC₀₋₈ (ng · h/g) | 547.6 | AUC₀₋₈ (ng · h/g) | 30873.6 |
| AUC₀₋₂₄ (ng · h/g) | 2408.5 | AUC₀₋₂₄ (ng · h/g) | 68428.0 |
| AUC₀₋ₗₐₛₜ (ng · h/g) | 3067.0 | AUC₀₋ₗₐₛₜ (ng · h/g) | 70489.0 |
| AUC_{0-inf} (ng · h/g) | 6349.0 | AUC_{0-inf} (ng · h/g) | 71366.7 |
| MRT₀₋ₗₐₛₜ (h) | 17.0 | MRT₀₋ₗₐₛₜ (h) | 11.0 |
| MRT_{0-inf} (h) | 48.2 | MRT_{0-inf} (h) | 11.3 |
| AUC_{Extra} (%) | 51.7 | AUC_{Extra} (%) | 1.2 |
| AUMC_{Extra} (%) | 82.9 | AUMC_{Extra} (%) | 4.2 |

Further analysis revealed the maximum concentration (Cmax) achieved by DA45 in both brain and plasma, with values recorded at 154 ng/g in brain tissue and 8105 ng/ml in plasma, respectively. The Cmax observed in the brain, coupled with the sustained half-life, highlights the excellent brain permeability of cyclic DA45. This is particularly significant as effective penetration of the blood-brain barrier (BBB) is a critical factor for therapeutic agents targeting CNS-related disorders

Moreover, the data suggest that cyclic DA45 not only penetrates the brain efficiently but also maintains therapeutic concentrations over an extended period, which could be advantageous in the context of chronic neurodegenerative diseases where prolonged drug action is desirable. These findings underscore the potential of cyclic DA45, and the other embodiments disclosed and predicted herein, as promising therapeutics for CNS-related disorders, particularly those involving ATAD3A oligomerization and associated neuropathologies.

As the above data clearly show, the enhanced metabolic stability and brain permeability of DA44 and DA45 extend their potential use to chronic neurodegenerative conditions, where sustained therapeutic action is necessary. The favorable pharmacokinetic profiles of these cyclic analogs suggest that they could maintain therapeutic levels in the CNS over extended periods, reducing the frequency of dosing and potentially improving patient compliance. This is particularly important in the context of diseases like PD and ALS, where long-term management of symptoms and neuroprotection are critical.

Further additional tests were performed in AD neurons, where neuronal porosome reconstitution combined with either a linear or circular peptide inhibitor of mitochondrial fission, was able to restore AD neurons to near normal levels of viability and greatly reduce oxidative stress in mitochondria. These additional tests were performed with the following Materials and Methods.

### Mouse HT-22 and Neuro2a Wt, Neuro2a APPwt and Neuro2a APPswe Cell Cultures

Mouse hippocampal HT-22 cells (MilliporeSigma, SCC129) and mouse neuroblastoma cell line Neuro2a cells (ATCC, CCL-131) were cultured in DMEM supplemented with 10% (v/v) heat-inactivated FBS and 1% (v/v) antibiotics (100 unit/mL penicillin, 100 μg/mL streptomycin). Neuro2a cells stably overexpressing human APP wildtype (APPwt) or Swedish mutant (APPswe, K670N and M671L APP, clone Swe.10) obtained from Dr. Gopal Thinakaran (University of Chicago), were cultured as described above. Cells were plated on poly-D-lysine/laminin (P6407, Sigma-Aldrich)-coated culture plates with or without coverslips at an appropriate cell density. All cells were maintained at 37° C. and 5% CO₂.

Porosome Isolated from Neuro2a Wt (Control) for Western Blot Analysis and for Reconstitution into Alzheimer's (Experimental) Neuro2a APPwt, and Neuro2a APPswe Cells

Neuro2a wt cells were used to isolate porosomes for reconstitution. SNAP-25 or Syntaxin-1a specific antibody (Abcam Cat. No. ab109105 and Ab41453 respectively, Cambridge, UK) was used to immunoisolate porosomes from solubilized cells. Cells were solubilized in Triton/Lubrol solubilization buffer (0.5% Lubrol; 1 mM benzamidine; 5 mM ATP; 5 mM EDTA; 0.5% Triton X-100, in PBS), supplemented with protease inhibitor mix (Sigma, St. Louis, MO). Protein in all fractions was estimated using BCA Protein assay Kit (ThermoFisher Cat. No. 23227, Rockford, IL 61101, USA). SNAP-25 specific antibody-crosslinked to protein A/G Magnetic-agarose (ThermoFisher Cat. No. 78609) was used. To reduce the antibody contamination in eluted protein solution, the antibody was chemically crosslinked to the agarose-magnetic beads. Briefly, the beads were resuspended in dilution buffer (1:1 ratio, 1 mg/mL BSA in PBS) for 10 min at 4° C., centrifuged for 1 min at 14,000 rpm in a bench top centrifuge, and the supernatant aspirated. SNAP-25 or Syntaxin-1a antibody (1 μg/mL) in dilution buffer was added to the beads at 1:1 ratio and mixed gently for 1 hr at 4° C. The beads were then washed twice with 10 volumes of dilution buffer. Dimethyl pimelimidate solution (DMP, 13 mg/ml, Sigma Cat. No. D8388) in wash buffer (0.2 M triethanolamine in PBS, Sigma Cat. No. 90279) was added to the SNAP-25 antibody conjugated beads (1:1 ratio) and resuspended for 30 min at room temperature (RT). The beads were then washed with wash buffer three time. (30 min/wash at RT), and resuspended in quenching buffer (50 mM ethanolamine in PBS, Sigma Cat. No. E0135) for 5 min at RT and washed with PBS twice. To remove excess unlinked antibody, the beads were washed with 1 M glycine pH 3, twice (10 min/wash at RT). Prior to use for immunoprecipitation, the beads were washed in PBS-TWEEN buffer three times. SNAP-25 antibody-crosslinked to the protein A/G Magnetic-agarose was incubated with the solubilized cell lysates for 16 h at 4° C. followed by washing with wash buffer (500 mM NaCl, 10 mM TRIS, 2 mM EDTA, pH 7.5). The immune-isolated porosomes associated with the immuno-agarose beads were dissociated and eluted using pH 3.0 PBS solution, and the eluted sample was immediately returned to neutral pH prior to Western Blot analysis and reconstitution assays. Porosome reconstitution into Neuro2a APPwt, and Neuro2a APPswe cells in culture was achieved by exposing 0.125 μg/mL porosomes isolated from Neuro2a wt cells.

### Plasmids and Transfection

Human ATAD3A-WT-Flag and ATAD3A-AN50-Flag plasmids were prepared using known techniques. Cells were transfected with plasmids using TransIT®-2020 transfection reagent (MIR5406, Mirus Bio LLC, Madison, WI), according to the manufacturer's instructions. Lenti-Syn-CYP46A1-mCherrypuro plasmid and Lenti-mCherry control plasmids were obtained from Vector-Builder Inc. Lentiviruses were generated by transfecting human embryonic kidney 293T (HEK293T) cells with plasmids encoding the envelope (pCMV-VSV-G; catalog no. 8454, Addgene), packing (psPAX2; catalog no. 12260, Addgene), and targeted open reading frame. The medium was changed 12 h after transfection, and the lentiviruses were harvested after 36 h. The lentiviruses were diluted with the corresponding medium at a 1:1 ratio, and the cells of interest were infected in the presence of Polybrene (8 μg/mL, Sigma-Aldrich) for 48 h. For knockdown of ATAD3A in Neuro2a cells, cells were infected with lentivirus of control shRNA and ATAD3A shRNAs (Sigma, TRCN0000242003 and TRCN0000241479), using known techniques.

### Control TAT and DA1 Peptides

Control peptide TAT and linear DA1 peptide (Product number P103882, Lot #OP082714SF-01) were synthesized at Ontores (Hangzhou, China). Their purities were assessed as >90% by mass spectrometry. Lyophilized peptides were dissolved in sterile water and stored at −80° C. until use. Linear DA1 is the first and only inhibitor that selectively binds to ATAD3A, reducing oligomerization, neuropathology, and behavioral deficits in AD mice without affecting wild-type counterparts. The peptide is particularly effective under stress or disease conditions, where oligomers accumulate and recruit Drp1. Thus, it is believed that DA1-like agents offer a new therapeutic approach.

### Cycilic DA1 Peptide Analog

In addition to the DA1 linear peptide, embodiments disclose herein use developed cyclic DA1 analogs with enhanced metabolic stability and improved on-target efficacy. Following peptide optimization and biochemical screening in AD cell cultures, cyclic DA1 analog DW45 was identified. DW45 displayed notable stability with a half-life of >289 minutes in a mouse plasma stability assay and >216 minutes in a hepatocyte stability assay, respectively. Moreover, the analogs presented herein (see Table 1) generally demonstrate stronger efficacy and on-target engagement in AD neuronal cultures compared to the linear DA1 peptide.

### MTT Assay

MTT (3-(4,5-dimethylthiazol-2-yl)-2,5-diphenyltetrazolium bromide) assay is a colorimetric method that measures cell viability, proliferation, and cytotoxicity. It is based on the reduction of a yellow tetrazolium dye, MTT, into purple formazan crystals by metabolically active cells. Abeta: Ameloid beta; TAT: Transactivator of transcription; DA1 (linear peptide); WX45 (DA1 cyclic peptide)

### Western Blot Analysis on Cell Homogenates and Isolated Porosomes

Total cell homogenates (TH) and porosomes immuno-isolated (IP) from expanded WT (Control) mouse neuroblastoma cell culture and experimental Alzheimer's WT-APP and Sew-APP cultures were subjected to SDS-PAGE and Western Blot analysis. 20 μg of proteins (TH) and 10 ng of isolated neuronal porosomes in Laemmli buffer were resolved in a 12.5% SDS-PAGE, followed by electrotransfer to 0.2-mm nitrocellulose (NC) membrane. The NC membrane was incubated for 1 hour at RT in blocking buffer (5% nonfat milk in PBS [pH 7.4] containing 0.1% Triton X-100 and 0.02% NaN₃) and immunoblotted for 2 hours at RT with antibodies to SNAP-25 (1:1000, Abcam Cat. No. ab 109105) and GAPDH (1:3,000, Santa Cruz Cat. No. sc-25778, Dallas, TX 75200, USA). Anti-rabbit horseradish peroxidase secondary antibody conjugates were used (1:5,000, Cell Signaling Technology Cat. No. 7074), followed by the NC membrane developed using Western Lightning Plus-ECL (PerkinElmer, Waltham, MA 02451. USA) and imaged using ChemiDoc XRT+ image system (Bio-Rad, Richmond, CA94806, USA).

### Immunofluorescence Cytochemistry

Cells were grown on coverslips, fixed with 4% paraformaldehyde for 20 min at room temperature, permeabilized with 0.1% Triton X-100 in PBS, and blocked with 2% normal goat serum. The cells were then incubated with the indicated primary antibodies overnight at 4° C. After washing with PBS, the cells were incubated with Alexa Fluor 488/568 or 405/568 secondary antibody (1:500; Thermo Fisher Scientific) for 2 h at room temperature. The nuclei were counterstained with DAPI (1:10,000; Sigma-Aldrich). Images of the staining were acquired using a Fluoview FV 1000 confocal microscope (Olympus).

The strategies employed in this disclosure, and the exemplary embodiments, including the incorporation of D-amino acids and the use of diverse cyclization techniques, provide a blueprint for developing other macrocyclic peptides with improved drug-like properties. These methodologies could be adapted to target a wide range of intracellular interactions relevant to CNS disorders, thereby broadening the scope of therapeutic interventions.

### Results and Discussion—Second Tests

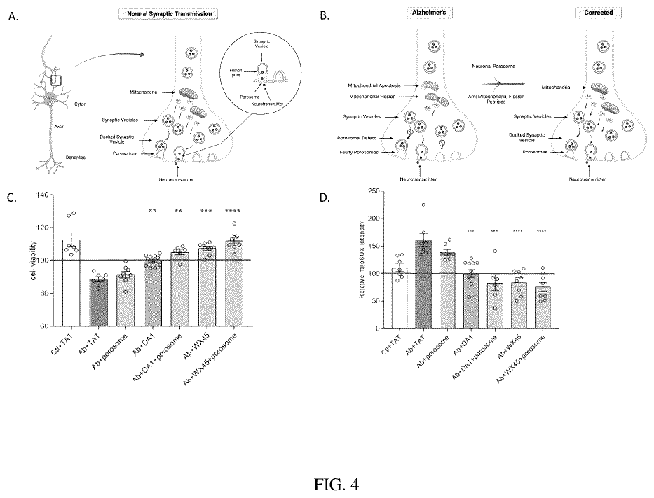

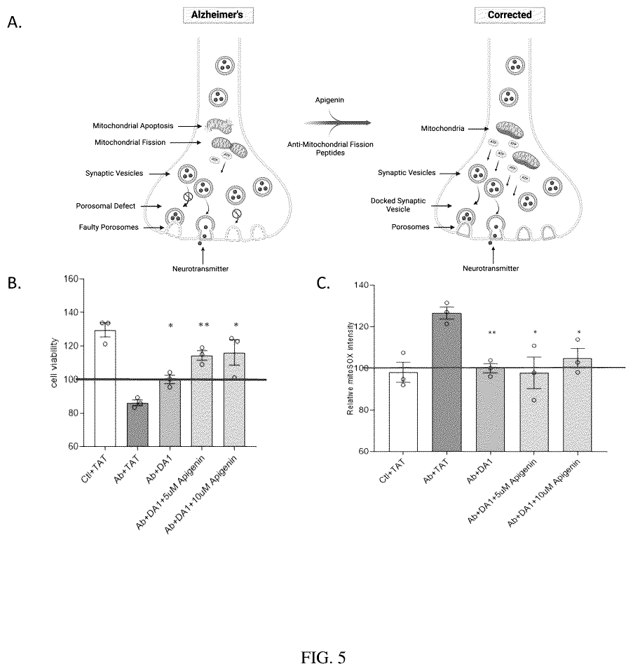

As seen herein and exemplified in FIGS. 4 and 5, both the linear and circular DA1 peptide (WX45) similarly demonstrated partial rescue from AD both in cell viability and in reduced mitochondrial superoxidase activity (mitoSOX) in Alzheimer's neurons (Ab+TAT). The WX45 circular DA1 peptide is demonstrated as more effective than the linear DA1 peptide. However, the neuronal porosome reconstitution combined with the DA1 peptide, especially the circular WX45 peptide inhibitor of mitochondrial fission, was able to restore AD neurons to near normal levels of viability and greatly reduce oxidative stress in mitochondria, demonstrating therapeutic application.

Additionally, the peptide and Apigenin, a small molecule flavonoid considered safe by the food and drug administration (FDA) and used as a food additive, was also able to greatly increase viability in AD neurons and reduce to near normal levels, mitoSOX (FIGS. 4 and 5) also demonstrating its use in AD therapy. Collectively, these results demonstrate the combinatorial use of the porosome reconstitution secretory reprogramming and the DA1 peptide and Apigenin metabolic correctors, as potential highly safe and effective AD therapies.

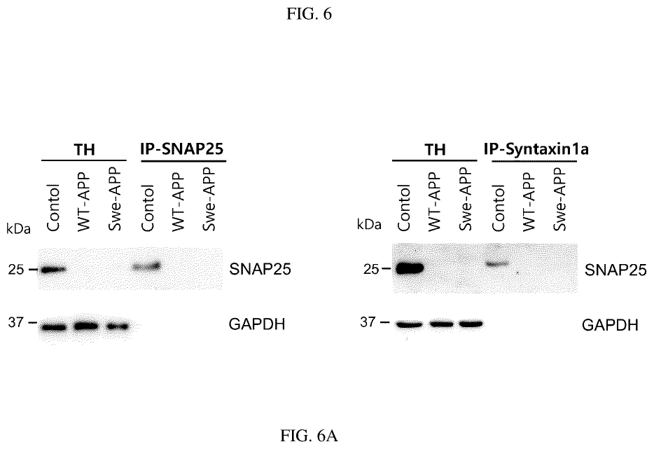

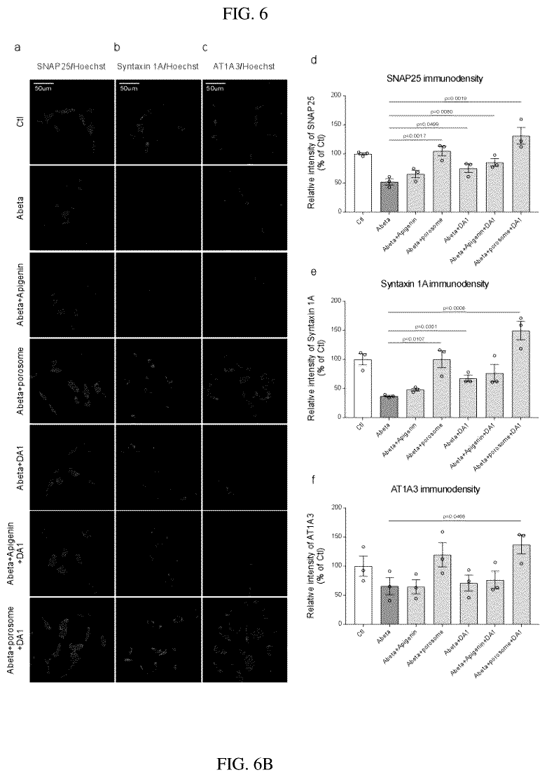

In agreement, as seen in FIG. 6, immunoblot analysis (FIG. 6A) and immunocytochemistry study (FIG. 6B) demonstrate depletion of porosome proteins especially the t-SNARE proteins SNAP-25 and Syntaxin-1A, which are restored following peptide and porosome-reconstitution therapy. For FIG. 6A, note that in Alzheimer's neurons (WT-APP and Swe-APP), there is undetectable levels of SNAP-25 both in total homogenates and in the immunoisolated porosome complex, immunoisolated using the t-SNARE SNAP-25 or Syntaxin-1a antibody.

Seen in FIG. 6B (with associated sub-figures a-f), immunocytochemistry demonstrates the depletion of porosome proteins SNAP-25, Syntaxin-1A, and Na⁺/K⁺ Transporting ATPase alpha 3 (ATIA3) in Alzheimer's neurons WT-APP (Abeta), which is fully restored to normal levels following porosome-reconstitution therapy, and partially by the DA1 peptide. The combined exposure to both the porosome and DA1 peptide, further enhances the expression of all three porosome proteins SNAP-25, Syntaxin-1A, and AT1A3. Modest increase in SNAP-25, Syntaxin-1A, or AT1A3 immunoreactivity is observed following exposure of Apigenin to the Alzheimer's neurons (+Abeta). Note that Apigenin and DA1 combination has a significant effect on enhancing expression of all three porosome proteins. (FIG. 6B, a) In this panel, the immunolocalization of SNAP-25 in untreated control neurons (Ctl), Abeta (Alzheimer's) neurons, Abeta neurons treated with Apigenin (Abeta+Apigenin), Abeta neurons reconstituted with neuronal porosomes, Abeta treated with the DA1 peptide, Abeta treated with Apigenin and the DA1 peptide, and Abeta treated with porosome and the DA1 peptide, is shown. (FIG. 6B, b) In this panel, the immunolocalization of Syntaxin-1A in untreated control neurons (Ctl), Abeta (Alzheimer's) neurons, Abeta neurons treated with Apigenin (Abeta+Apigenin), Abeta neurons reconstituted with neuronal porosomes, Abeta treated with the DA1 peptide, Abeta treated with Apigenin and the DA1 peptide, and Abeta treated with porosome and the DA1 peptide, is shown. (FIG. 6B, c) In this panel, the immunolocalization of AT1A3 in untreated control neurons (Ctl), Abeta (Alzheimer's) neurons, Abeta neurons treated with Apigenin (Abeta+Apigenin), Abeta neurons reconstituted with neuronal porosomes, Abeta treated with the DA1 peptide, Abeta treated with Apigenin and the DA1 peptide, and Abeta treated with porosome and the DA1 peptide, is shown. (FIG. 6B, d) In this bar graph, the quantification of the immunolocalization of SNAP-25 in untreated control neurons (Ctl), Abeta (Alzheimer's) neurons, Abeta neurons treated with Apigenin (Abeta+Apigenin), Abeta neurons reconstituted with neuronal porosomes, Abeta treated with the DA1 peptide, Abeta treated with Apigenin and the DA1 peptide, and Abeta treated with porosome and the DA1 peptide, is shown. (FIG. 6B, c) In this bar graph, the quantification of the immunolocalization of Syntaxin-1A in untreated control neurons (Ctl), Abeta (Alzheimer's) neurons, Abeta neurons treated with Apigenin (Abeta+Apigenin), Abeta neurons reconstituted with neuronal porosomes, Abeta treated with the DA1 peptide, Abeta treated with Apigenin and the DA1 peptide, and Abeta treated with porosome and the DA1 peptide, is shown. (FIG. 6B, f) In this bar graph, the quantification of the immunolocalization of AT1A3 in untreated control neurons (Ctl), Abeta (Alzheimer's) neurons, Abeta neurons treated with Apigenin (Abeta+Apigenin), Abeta neurons reconstituted with neuronal porosomes, Abeta treated with the DA1 peptide, Abeta treated with Apigenin and the DA1 peptide, and Abeta treated with porosome and the DA1 peptide, is shown.

Consequently, in Alzheimer's patients there is an increase in levels of SNAP-25 in the cerebrospinal fluid, which has been associated with cognitive decline. Upon porosome reconstitution in APP neurons, as seen in FIG. 6, the immunocytochemistry demonstrates the restoration of SNAP-25 and other porosome-associated proteins, that result in increased cell viability to near normal levels and a decrease in mitoSOX activity (FIG. 4).

In early 2023, there were 187 Phase 1, 2, and 3 clinical trials assessing 141 drugs for AD. Thirty-six drugs were being assessed in Phase 3, 87 in Phase 2, and 31 in Phase 1. Although, neurotransmitter receptors, amyloid plaque, synaptic function, and inflammation were the most common targets of drugs in the pipeline, most therapies have focused on clearing the buildup of amyloid plaques and on reducing inflammation. While these are important advancements in AD therapy, they are primarily focused as treatments to ameliorate the various consequences of the disease; there are no cures in sight, nor are therapies developed that treat the disease at the very early stage.

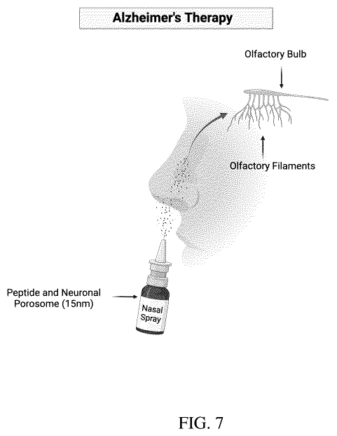

As presented herein, the systems level approach in correcting both the metabolic and secretory defects in AD at early stages of the disease, shows great promise. It is important to note that in >90% of Alzheimer's patients, olfactory dysfunction precedes cognitive decline. Moreover, the olfactory bulb of AD mice demonstrate loss in expression of SNAP-25. Since the olfactory bulb is connected via nerves to the brain centers involved in learning, memory and emotion, suggests that the disclosed combinatorial DA1 peptide, either with Apigenin or porosome, would be a viable and highly effective AD therapy, deliverable through the nasal route, especially at the early stages of the disease. (FIG. 7)

In this regard, while the DA1 circular peptide and Apigenin are readily available, the scale-up of the neuronal porosome biologic will be achieved as envisioned by immunoisolation from 3D cultures of the human neuroblastoma SH-SY5Y cell line grown in biorcactors. 3D neuronal cultures may be used since the capacity of a neuron to establish synaptic connections is an essential prerequisite for its functional maturation, and that the formation of synapse involves the appropriate assembly of the porosome secretory machinery at the nerve terminal (FIG. 4, A). Since the neuronal porosome is present in all neurons, the above-mentioned scale-up approach offers reproducibility, reliability and safety. One needs to be critically aware that, since several of the nearly 30 or so proteins of the porosome complex are impacted following a mutation or absence of even one of the 30 or so porosome proteins, the resulting impairment in secretion, can effectively ameliorated by reintroducing all the impacted porosome proteins within the secretory complex, a near-impossible undertaking, which could be corrected by introducing normal neuronal porosomes at the pre-synaptic membrane. Porosome reconstitution therapy therefore remains the most optimal approach in ameliorating neurotransmitter release defects in AD. The normal secretion of neurotransmitter and its timely breakdown is physiologically relevant and optimal, rather than the available AD medications such as cholinesterase inhibitors that prevent the breakdown of acetylcholine in the brain to improve neuronal communication. However, cholinesterase inhibitor drugs exhibit side effects that may include nausea, vomiting, diarrhea, muscle cramps, fatigue and even weight loss.

### EXAMPLES

### Example 1

In an envisioned embodiment, a composition comprising flavonoids alone, or in a pharmaceutical preparation, are administered through the nasal olfactory route to a subject diagnosed with a neural disease (e.g., AD, Huntington's, ALS, etc.). The flavonoid may be admixed with a pharmaceutically acceptable excipient to form a nanoemulsion.

### Example 2

In certain other envisioned embodiments, a macrocyclic peptide targeting a mitochondrial process is administered to a subject. The subject may be experiencing a neural disease or may be diagnosed as at-risk of a neural disease. The macrocyclic peptide may be administered through the nasal olfactory route. The macrocyclic peptide may be administered alone or in conjunction with one or more flavonoid molecules. In certain embodiments the flavonoid is apigenin. In still other embodiments, the subject is administered the flavonoid and macrocyclic peptide mixed with a pharmaceutically acceptable excipient. In still other envisioned embodiments, the mixture is part of a nanoemulsion administered via the nasal olfactory route. Although a nanoemulsion may be a preferred embodiment, other methods of delivery, such as those discussed herein, are also possible and may be preferred given an evaluated state of a presenting subject.

### Example 3

In certain other envisioned embodiments, one or more macrocyclic peptides are administered to a subject experiencing or at risk of contracting a neurological disease. In certain embodiments the macrocyclic peptides are administered in conjunction with one or more flavonoid molecules. In certain embodiments the macrocyclic peptides are DA44 and DA45 either alone or in combination with each other and/or additional peptides. In certain embodiments the flavonoid molecule is apigenin.

One or more of peptides of the therapeutic peptides described herein can also be modified by natural processes, such as posttranslational processing, and/or by chemical modification techniques, which are known in the art. Modifications may occur in the peptide including the peptide backbone, the amino acid side-chains and the amino or carboxy termini. It will be appreciated that the same type of modification may be present in the same or varying degrees at several sites in a given peptide. Modifications comprise for example, without limitation, acetylation, acylation, addition of acetomidomethyl (Acm) group, ADP-ribosylation, amidation, covalent attachment to fiavin, covalent attachment to a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cystine, formation of pyroglutamate, formylation, gamma-carboxylation, glycosylation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation and ubiquitination (for reference see, Protein-structure and molecular properties, 2nd Ed., T. E. Creighton, W. H. Freeman and Company, New-York, 1993).

Peptides and/or proteins described herein may also include, for example, biologically active mutants, variants, fragments, chimeras, and analogues; fragments encompass amino acid sequences having truncations of one or more amino acids, wherein the truncation may originate from the amino terminus (N-terminus), carboxy terminus (C-terminus), or from the interior of the protein. Analogues of the invention involve an insertion or a substitution of one or more amino acids. Variants, mutants, fragments, chimeras, and analogues may function as inhibitors of the interaction of Drp1 and ATAD3A (without being restricted to the present examples).

The therapeutic peptides described herein may be prepared by methods known to those skilled in the art. The peptides and/or proteins may be prepared using recombinant DNA. For example, one preparation can include cultivating a host cell (bacterial or eukaryotic) under conditions, which provide for the expression of peptides and/or proteins within the cell.

The purification of the polypeptides may be done by affinity methods, ion exchange chromatography, size exclusion chromatography, hydrophobicity or other purification technique typically used for protein purification. The purification step can be performed under non-denaturating conditions. On the other hand, if a denaturating step is required, the protein may be renatured using techniques known in the art.

In some embodiments, the therapeutic peptides described herein can include additional residues that may be added at either terminus of a polypeptide for the purpose of providing a “linker” by which the polypeptides can be conveniently linked and/or affixed to other polypeptides, proteins, detectable moieties, labels, solid matrices, or carriers.

Amino acid residue linkers are usually at least one residue and can be 40 or more residues, more often 1 to 10 residues. Typical amino acid residues used for linking are glycine, tyrosine, cysteine, lysine, glutamic and aspartic acid, or the like. In addition, a subject polypeptide can differ by the sequence being modified by terminal-NH2 acylation, e.g., acetylation, or thioglycolic acid amidation, by terminal-carboxylamidation, e.g., with ammonia, methylamine, and the like terminal modifications. Terminal modifications are useful, as is well known, to reduce susceptibility by proteinase digestion, and therefore serve to prolong half-life of the polypeptides in solutions, particularly biological fluids where proteases may be present. In this regard, polypeptide cyclization is also a useful terminal modification, and is particularly preferred also because of the stable structures formed by cyclization and in view of the biological activities observed for such cyclic peptides as described herein.

In some embodiments, the linker can be a flexible peptide linker that links the therapeutic peptide to other polypeptides, proteins, and/or molecules, such as detectable moieties, labels, solid matrices, or carriers. A flexible peptide linker can be about 20 or fewer amino acids in length. For example, a peptide linker can contain about 12 or fewer amino acid residues, e.g., 3, 4, 5, 6, 7, 8, 9, 10, 11, and 12. In some cases, a peptide linker comprises two or more of the following amino acids: glycine, serine, alanine, and threonine.

The therapeutic agents described herein may be modified (e.g., chemically modified). Such modification may be designed to facilitate manipulation or purification of the molecule, to increase solubility of the molecule, to facilitate administration, targeting to the desired location, to increase or decrease half-life. A number of such modifications are known in the art and can be applied by the skilled practitioner.

In the methods of treatment disclosed herein, a therapeutically effective amount of the therapeutic agent is administered to the subject to treat a disorder or mitochondrial disorder, such as a neurodegenerative disease. In one embodiment, a formulation including the therapeutic agent can be administered to the subject systemically in the period from the time of, for example, up to hours, days, and/or weeks after the disease or disorder is diagnosed.

The therapeutic agents can be delivered to a subject by any suitable route, including, for example, local and/or systemic administration. Systemic administration can include, for example, parenteral administration, such as intramuscular, intravenous, intraarticular, intraarterial, intrathecal, subcutaneous, or intraperitoneal administration. The agent can also be administered orally, transdermally, topically, by inhalation (e.g., intrabronchial, intranasal, oral inhalation or intranasal drops) or rectally. In some embodiments, the therapeutic agent can be administered to the subject via intravenous administration using an infusion pump to deliver daily, weekly, or doses of the therapeutic agent.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein without any additional undue experimentation. It being understood that various changes may be made in the function and arrangement of elements described in an exemplary embodiment without departing from the scope of the various embodiments as set forth in the appended claims.

Since certain changes may be made in the above-described disclosure, without departing from the spirit and scope of the disclosure herein involved, it is intended that all of the subject matter of the above description shown in the accompanying drawings shall be interpreted merely as examples illustrating the inventive concept herein and shall not be construed as limiting the disclosure.

Finally, the written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A composition comprising:
a pharmaceutically acceptable preparation of a flavonoid and at least one macrocyclic peptide associated with mitochondrial fission.

2. The composition of claim 1 wherein the composition is a nanoemulsion for nasal administration.

3. The composition of claim 1 wherein the at least one peptide encodes a Drp1 adaptor protein or part thereof.

4. The composition of claim 1 wherein the macrocyclic peptide is based on DA1.

5. The composition of claim 4 wherein the macrocyclic peptide is DA44 or DA45.

6. The composition of claim 4 further comprising one or more additional peptides.

7. The composition of claim 6 wherein the one or more additional peptides are selected from the group of: DA44, DA45, linear DA1, and combinations thereof.

8. The composition of claim 1 wherein the flavonoid is apigenin.

9. A composition, comprising:
apigenin; and,
at least one DA1 based macrocyclic peptide.

10. The composition of claim 9 wherein the at least one macrocyclic peptide is DA44 or DA45.

11. The composition of claim 9 wherein the composition further comprises at least one additional pharmaceutical excipient.

12. The composition of claim 11 wherein the composition is prepared in the form of a nanoemulsion.

13. The composition of claim 11 wherein the composition is prepared in the form of a suspended mist, powder, or solution.

14. A method, comprising:
administering to a subject the composition of claim 1.
